(19) United States
(12) Patent Application Publication
(10) Pub. No.: US 2025/0012795 A1
(43) Pub. Date: Jan. 9, 2025

(54) DIAGNOSING MULTIPLE SCLEROSIS (MS)

(71) Applicant: The Regents of the University of Colorado, A Body Corporate, Denver, CO (US)

(72) Inventors: Xiaoli Yu, Denver, CO (US); Michael Graner, Bennett (CO); Wenbo Zhou, Denver, CO (US); Timothy Vollmer, Aurora, CO (US); Enrique Alvarez, Aurora, CO (US)

(21) Appl. No.: 18/697,411

(22) PCT Filed: Sep. 29, 2022
(86) PCT No.: PCT/US2022/077300

§ 371 Date: Mar. 29, 2024

Related U.S. Application Data
(60) Provisional application No. 63/249,741, filed on Sep. 29, 2021.

(51) Int. Cl. G01N 33/564

(52) U.S. Cl. CPC G01N 33/564; G01N 2800/285

(57) ABSTRACT

Compositions and methods for diagnosing and treating multiple sclerosis (MS) and other health conditions involve collecting serum samples from a subject and exposing the serum samples to a protein capture composition, collecting the unbound eluent or flow-through, and measuring the levels of at least one immunoglobulin therein. Methods for differentiating between different types of MS also involve exposing serum samples to a protein capture composition and detecting immunoglobulin levels in the flow-through. Based on the detected immunoglobulin levels, an MS subtype is diagnosed in a subject and a treatment regimen can be adjusted or ceased according to the diagnosis.

### CROSS REFERENCE TO RELATED APPLICATIONS

This application is a national phase entry of International Patent Application No. PCT/US2022/077300, filed Sep. 29, 2022, entitled “DIAGNOSING MULTIPLE SCLEROSIS (MS),” which in turn claims priority to U.S. Provisional Patent Application No. 63/249,741, filed Sep. 29, 2021, entitled “METHODS. COMPOSITIONS, AND APPLICATIONS FOR DIAGNOSING MULTIPLE SCLEROSIS (MS)”, each of which is incorporated by reference herein, in the entirety and for all purposes.

### GOVERNMENT FUNDING

This invention was made with government support under grant number 4R33MH118174-03 from NIH. The government has certain rights in the invention.

### FIELD

Embodiments of the present disclosure generally relate to compositions and methods for diagnosing and treating multiple sclerosis (MS) and other health conditions. In certain embodiments, compositions and methods are disclosed for differentiating between different types of MS.

### BACKGROUND

Multiple sclerosis (MS), also known as encephalomyelitis disseminata, is a demyelinating condition where insulating coverings of nerve cells in the brain and spinal cord become damaged. This damage disrupts the nervous system's ability to transmit signals, resulting in a range of complications, including physical, mental, and sometimes psychiatric issues. Specific symptoms can include double vision, blindness in one eye, muscle weakness, and trouble with sensation or coordination. MS takes several forms, with new symptoms either occurring in isolated attacks (relapsing forms) or building up over time (progressive forms) of MS. Some or all symptoms can disappear between flare-ups, but permanent neurological problems often remain, especially as the condition worsens and relapses intensify.

MS has no cure but, it is the most common immune-mediated disorder affecting the central nervous system. The condition usually begins between the ages of twenty and fifty and is twice as common in women as in men. Diagnosing MS quickly is crucial to facilitate intervention and be able to effectively treat the symptoms and eventually the condition itself. A need exists for more rapid and reliable diagnosis of MS and its subtypes.

### SUMMARY

Embodiments of the present disclosure generally relate to compositions and methods for diagnosing and treating multiple sclerosis (MS), MS subtypes, and other health conditions. In certain embodiments, compositions and methods are disclosed for differentiating between different types of MS. In certain embodiments, serum from a subject can be used as a sample instead of spinal fluid to diagnose MS and/or an MS subtype. In accordance with these embodiments, one or more serum or plasma samples can be obtained from a subject having or suspected of having or developing MS and processed for analysis. In some embodiments, serum samples can be analyzed for the presence of and/or the concentration of IgG antibodies after processing, including the concentration of total immunoglobulin G (“IgG”), immunoglobulin G1 (“IgG1”), and immunoglobulin G3 (“IgG3”). It is understood that directly assaying serum samples from a subject for IgG antibodies has been ineffective for diagnosing MS, MS progression, and MS subtypes, and for monitoring MS treatment response. Compositions and methods disclosed herein alleviate this issue, providing less invasive and improved methods for MS diagnosis and typing. In some embodiments, methods disclosed herein alleviate the need to obtain and test spinal fluid from a subject.

In some embodiments, serum samples can be obtained from a subject having, suspected of developing, or currently developing MS. In certain embodiments, samples are evaluated for the presence and/or concentration of IgG antibodies. In accordance with these embodiments, samples can be analyzed for the presence and/or concentration of IgG antibodies in one or more serum sample(s) having undergone exposure to a capture composition, e.g., protein-coated matrix, where the flow-through of the capture composition can be analyzed. In certain embodiments, one or more of total IgG, IgG1 and/or IgG3 antibodies can be analyzed in the flow-through of the capture composition in order to diagnose MS and/or an MS subtype in a subject. In some embodiments, serum samples can be exposed to Protein A to permit interaction with this agent. Protein A is a cell-wall protein of Staphylococcus aureus that binds with high affinity to the Fc region of immunoglobulins from various species. The bacterium uses Protein A as part of its defense against a host immune system. Recombinant Protein A can be generated using E. coli or other microorganisms for use and the manufacture of affinity chromatography media. IgG antibodies form larger aggregates which can activate complement. Furthermore, Protein A inhibits complement activation by interfering with IgG hexamer formation. In accordance with embodiments disclosed herein, non-binding serum components can be collected and analyzed for total IgG, IgG1 and/or IgG3 antibodies or concentration thereof in a sample. In other embodiments, flow-through or non-binding components can be analyzed by any method known in the art to capture IgG antibodies. In some embodiments, ELISA assays or other comparable assays can be used to measure the presence and/or concentration of one or more IgG antibodies derived from one or more serum samples.

In some embodiments, assays disclosed herein can be used to screen one or more serum samples of a subject to distinguish MS subtypes. In accordance with these embodiments, one or more serum samples can be collected from a subject and analyzed for the presence and/or concentration of IgG antibodies (e.g. IgG1 or IgG3) in order to distinguish whether the subject has RRMS (Relapse Remitting MS), SPMS (Secondary Progressive MS), or PPMS (Primary Progressive MS). In some embodiments, a subject can be diagnosed with a particular subtype and then treated with a standard agent or not treated based on the subtype. In accordance with these embodiments, a subject having SPMS may not respond to an agent being used to treat RRMS. In other embodiments, serum samples and serum sample analysis disclosed herein can be used to assess MS progression and/or efficacy of a treatment where IgG presence and/or concentration in one or more processed serum samples can be measured against control samples from a subject having a particular MS subtype and non-MS or healthy controls. In certain embodiments, methods disclosed herein can be used to assess efficacy as a screening process for new or experimental agents by processing and analyzing serum samples using compositions and methods of this disclosure from treated and untreated MS patients over a predetermined period.

In some embodiments, kits are contemplated for transport, storage and use of serum samples and/or assays disclosed herein to diagnose MS and/or distinguish an MS subtype. In some embodiments, kits may be configured for immediate analysis of one or more serum samples in order to reduce sample degradation.

In accordance with embodiments of the present disclosure, a processed serum sample composition may include a processed serum sample from a subject having Multiple Sclerosis (MS) with an enriched concentration of at least one IgG compared to a control processed serum sample or an unprocessed serum sample. The processed serum sample can also include a diluent, which may include one or more of PBS. TBS, or H₂O, and which may lack magnesium, calcium, or both.

In some embodiments, the processed serum sample may be a flow-through sample or unbound eluent remaining after exposure of an unprocessed serum sample to at least one of a Protein A matrix, a Protein G matrix, or a Protein A/Protein G matrix. In some embodiments, the enriched concentration of at least one IgG comprises more total IgG, IgG1 or IgG3 in the processed serum sample from the subject having MS compared to a control processed serum sample.

In accordance with embodiments of the present disclosure, a method for diagnosing a neurological disorder in a subject may involve obtaining one or more serum samples from a subject suspected of having or developing a neurological disorder, exposing the one or more serum samples to one or more of a Protein A matrix, a Protein G matrix, or Protein A/Protein G matrix, and collecting flow-through of the one or more serum samples after exposing the samples to the one or more of the Protein A matrix, the Protein G matrix, or the Protein A/Protein G matrix. The method may further involve measuring IgG levels in the flow-through of the one or more serum samples and diagnosing a neurological disorder in the subject based on at least one of a total IgG (H+L)/Fc level, an IgG1 level, or an IgG3 level in the flow-through of the one or more serum samples compared to one or more samples from a control subject not having a neurological disorder.

In some embodiments, the method may further involve diagnosing the subject with MS based on the IgG1 level in the flow-through of the one or more serum samples. In some embodiments, measuring IgG levels involves measuring at least one of IgG1 or IgG3 levels. In some embodiments, measuring IgG (H+L)/Fc levels comprises measuring total IgG (H+L)/Fc and comparing total IgG (H+L)/Fc to healthy control subject samples, and wherein elevated total IgG (H+L)/Fc levels compared to the healthy control subject samples is indicative of having a neurological disorder in the subject. In some embodiments, measuring IgG levels involves using an ELISA assay, flow cytometry, Nephelometry or other immunoassay for detecting IgG antibodies in the flow-through of the one or more serum samples. In some embodiments, the method distinguishes a subject having MS from a subject having a different inflammatory CNS disorder based on the level of IgG or the level of IgG1. In some embodiments, the one or more samples may be exposed to one or more of the Protein A matrix, the Protein G matrix, or the Protein A/Protein G matrix at least two times by collecting the flow-through and reapplying the flow-through to at least a second Protein A matrix, Protein G matrix, or Protein A/Protein G matrix. In some embodiments, the one or more serum samples may be exposed to a Protein A matrix and may further involve measuring IgG3 levels in the flow-through, where elevated IgG3 levels compared to healthy control samples and samples from a subject having a neurological disorder other than MS is indicative that the subject has MS. In some embodiments, the one or more serum samples are exposed to a Protein G matrix and the method further involves measuring IgG1 levels in the flow-through of the one or more serum samples, where elevated IgG1 levels compared to healthy control samples and samples from a subject having a neurological disorder other than MS is indicative that the subject has MS. In some embodiments, the method may further involve performing a cytotoxic analysis of one or more serum samples exposed to at least one of a Protein A or Protein G matrix and measuring apoptosis, where increased apoptosis in the samples is indicative of at least one of MS or MS progression. In some embodiments, a method may further involve exposing the flow-through to a filter having a molecular weight cut-off of about 110 kDa, about 200 kDa or about 300 kDa and measuring at least one of IgG1 and IgG4 levels in a retentate. Embodiments may further involve treating the subject to reduce IgG levels. In some embodiments, reducing IgG levels involves reducing IgG1, which may abolish or reduce neuronal toxicity observed in a subject having MS.

In accordance with embodiments of the present disclosure, a method for identifying subtypes of MS in a subject may involve obtaining one or more serum samples from a subject suspected of having or developing MS, exposing the one or more serum samples to a Protein A matrix, and collecting the flow-through of the one or more serum samples after exposing the samples to the Protein A matrix. The method may further involve measuring IgG levels in the flow-through of the one or more serum samples, comparing the IgG levels in the flow-through of the serum samples to IgG levels in flow-through of control samples, and diagnosing Relapsing-Remitting MS (RRMS), Secondary-Progressive MS (SPMS), or Primary-Progressive MS (PPMS) in the subject based on the IgG levels in the flow-through of the one or more serum samples. The method may further involve adjusting a treatment of the subject based on the diagnosis.

In some embodiments, measuring IgG levels involves measuring IgG1 levels. The method of such embodiments may further involve diagnosing the subject with Secondary-Progressive MS (SPMS) when the level of IgG1 is elevated compared to an IgG1 level of a control sample. Some embodiments may further involve analyzing the flow-through of the one or more serum samples for protein expression of one or more of IGKV1-5 (Immunoglobulin Kappa Variable 1-5); IGLV2-18 (Immunoglobulin Lambda Variable 2-18); C5 (Complement component 5); CFI (Complement Factor I); ORM1 (Orosomucoid 1); IGHV1-18 (Immunoglobulin Heavy Variable 1-18); IGHV3-49 (Immunoglobulin Heavy Variable 3-49); IGLV3-21 (Immunoglobulin Lambda Variable 3-21); LGALS3BP (Galectin 3 Binding Protein); PROC (Protein C, Inactivator Of Coagulation Factors Va And VIIIa); and SERPINAS (serine proteinase inhibitor). In some embodiments, the elevated IgG1 level in the flow-through of the one or more serum samples from the subject is about 1.5, about 2.0, about 2.5, or about 3.0 times greater than the IgG1 level of the control sample. In some embodiments, the elevated IgG1 level in the flow-through of the one or more serum samples is about 1.5, about 2.0, about 2.5, or about 3.0 times greater than an IgG1 level in an unprocessed serum sample. In some embodiments, the flow-through of the one or more serum samples is further subjected to mass spectrometry. Embodiments may further involve identifying clusters of differentially expressed proteins using proteomics data obtained from the mass spectrometry.

Higher total IgG and IgG1 may be detected by collections of retentates (not the filtrates) after filtration of MS plasma/serum directly with 300 kDa columns or 0.1 μm columns. Therefore, patients with MS may be identified and distinguished from subjects having other CNS disorders and healthy donors by directly measuring IgG antibodies in the retentates after filtrations of plasma or serum in accordance with embodiments disclosed herein.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of certain embodiments presented herein.

FIGS. 1A-1B represent exemplary experiments illustrating an IgG heavy chain band (75 kDa) found only in Protein A flow-through after exposure to exemplary binding matrices from samples of serum and cerebral spinal fluid from a subject having primary progressive MS in accordance with certain embodiments of the present disclosure.

FIGS. 2A-2D represent exemplary experiments illustrating higher levels of IgG1 and IgG3 detection in Protein A flow-through of serum from subjects having MS (n=8) compared to healthy controls (n=8) in accordance with certain embodiments of the present disclosure.

FIGS. 3A-3B represent exemplary experiments illustrating proteomic data of mass spectrometry analysis of flow-through sera samples obtained from relapse remitting MS (RRMS), secondary progressive MS (SPMS), and healthy control (HC) subjects in accordance with certain embodiments of the present disclosure. Enriched immunoglobulins and complements were detected in MS AFT.

FIGS. 4A-4B represent exemplary experiments illustrating an adapted designed assay for detecting IgG1 in Protein A flow-through of serum samples collected from control subjects. MS patients, patients having other neurological diseases (OND), and healthy controls in accordance with certain embodiments of the present disclosure.

FIGS. 5A-5B represent exemplary experiments illustrating an adapted designed assay for detecting IgG1 in Protein A flow-through of serum samples collected from control subjects with inflammatory CNS disorders (IC) and MS patients in accordance with certain embodiments of the present disclosure.

FIGS. 6A-6B represent exemplary experiments illustrating an adapted designed assay for detecting IgG1 in Protein A flow-through of serum samples collected from MS patients having one of three types of MS: RRMS (Relapse Remitting MS), SPMS (Secondary Progressive MS), or PPMS (Primary Progressive MS) in accordance with certain embodiments of the present disclosure.

FIG. 7 represents an exemplary experiment illustrating data from an assay for IgG1 in Protein A-purified IgG protein samples that were isolated from serum collected from healthy patients. MS patients, and patients having other neurological diseases in accordance with certain embodiments of the present disclosure.

FIGS. 8A-8B represent exemplary experiments illustrating data from an assay for IgG3 in Protein A flow-through of serum collected from healthy patients, MS patients, and patients having other neurological diseases and HC in accordance with certain embodiments of the present disclosure.

FIGS. 9A-9B represent exemplary experiments illustrating an assay for IgG1 and IgG3 in Protein G flow-through of serum collected from healthy patients. MS patients, and patients having other neurological diseases in accordance with certain embodiments of the present disclosure.

FIGS. 10A-10B represent exemplary experiments illustrating co-localization of IgG and an apoptosis marker in neuronal cell lines after treatment with MS serum Protein A flow-through (“A-FT”), where MS A-FT demonstrated significant neuronal apoptosis compared to all controls (OND and HC) in accordance with certain embodiments of the present disclosure.

FIGS. 11A-11D represent exemplary experiments illustrating neuronal cell death in newborn mouse brain slices after treatment with MS serum A-FT (compared to controls) in accordance with certain embodiments of the present disclosure.

FIGS. 12A-12B represent exemplary experiments illustrating that MS serum A-FT demonstrated higher IgG1 levels and higher cytotoxicity in neurons in SPMS compared to RRMS and PPMS, and in all MS compared to control serum A-FT (tumor patients) in accordance with certain embodiments of the present disclosure.

FIG. 13 represents an exemplary experiment illustrating higher total protein concentration in the 300 kDa retentates of SPMS serum A-FT compared to healthy control (HC) serum A-FT retentates in accordance with certain embodiments of the present disclosure.

FIG. 14 represents an exemplary experiment illustrating concentration of IgG subclass in the 300 kDa retentates of SPMS serum A-FT and HC serum A-FT retentates in accordance with certain embodiments of the present disclosure.

FIGS. 15A-15B represent exemplary experiments illustrating neurotoxicity of MS serum A-FT and HC serum A-FT 300 kDa retentates and filtrates in SH-SY5Y cells in accordance with certain embodiments of the present disclosure.

FIG. 16 represents an exemplary experiment illustrating cytotoxicity (apoptosis) tests of IgG subclass-depleted MS serum A-FT and HC serum A-FT in SH-SY5Y cells in accordance with certain embodiments of the present disclosure.

FIG. 17 represents an exemplary experiment illustrating reduced IgG1 levels in Protein A flow-through of serum collected from MS patients that had been previously treated with rituximab (RTX) and/or ocrelizumab (OCR) in accordance with certain embodiments of the present disclosure.

FIGS. 18A-18B represent exemplary experiments illustrating reduced level of neurotoxicity in neuroblastoma cells of MS serum A-FT collected from MS patients that had been previously treated with rituximab (RTX) and/or ocrelizumab (OCR) in accordance with certain embodiments of the present disclosure.

FIGS. 19A-19C represent exemplary experiments illustrating total IgG detection determined via ELISA assays of Protein A flow-through of plasma samples from subjects having 3 subtypes of MS compared to all types of controls [inflammatory CND diseases (IC), HC, non-inflammatory CNS diseases (NIC)] in accordance with certain embodiments of the present disclosure.

FIGS. 20A-20C represent exemplary experiments illustrating IgG1 detection determined via ELISA assays of Protein A flow-through of serum from subjects having different subtypes of MS compared to control samples in accordance with certain embodiments of the present disclosure. Data shows higher levels of IgG1 in SPMS compared to both RRMS and PPMS.

FIGS. 21A-21D represent exemplary experiments illustrating IgG aggregation in plasma-derived Protein A flow-through samples MS (top) and HC (bottom) examined via transmission electron microscopy in accordance with certain embodiments of the present disclosure.

FIGS. 22A-22D represent exemplary nanoparticle tracking experiments illustrating that 300 kDa retentates captured in Protein A flow-through derived from the serum of MS patients contained larger particles relative to healthy control Protein A flow-through. FIGS. 22E-22F represent exemplary experiments illustrating higher levels of aggregates present in MS compared with controls by nanoparticle tracking analysis (22E) and by protein aggregate analysis (22F) of Protein A flow-through samples obtained from subjects having MS and healthy controls in accordance with certain embodiments of the present disclosure.

FIGS. 23A-23C represent exemplary experiments illustrating that plasma retentates obtained from MS patients after 0.1 μm filtration contain higher levels of neuronal cytotoxicity, and higher protein aggregates that include elevated IgG1 levels and cause increased neuronal cytotoxicity in accordance with certain embodiments of the present disclosure.

FIGS. 24A-24C represent exemplary experiments illustrating that higher levels of total protein are present in the retentate of 0.1 μm filtration of MS plasma samples in accordance with certain embodiments of the present disclosure.

FIGS. 25A-25H represent exemplary embodiments illustrating that custom ELISA protocols disclosed herein and commercially available ELISA kits both detected significantly elevated levels of IgG1 and IgG3 in Protein A flow-through samples derived from subjects afflicted with MS in accordance with certain embodiments of the present disclosure.

FIGS. 26A-26F represent exemplary embodiments illustrating that age may correlate with IgG levels present in serum-derived samples obtained from patients afflicted with MS, but in control samples, age has no impact for IgG levels.

### DEFINITIONS

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, the terms “treat,” “treating,” “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

### DETAILED DESCRIPTION

In the following sections, certain exemplary compositions and methods are described in order to detail certain embodiments of the disclosure. It will be obvious to one skilled in the art that practicing the certain embodiments does not require the employment of all or even some of the specific details outlined herein, but rather that concentrations, times and other specific details can be modified through routine experimentation. In some cases, well-known methods or components have not been included in the description. Embodiments of the present disclosure generally relate to compositions, reagents, kits, and methods for diagnosing, monitoring and treating multiple sclerosis (MS), MS subtypes, and other health conditions. In certain embodiments, compositions, reagents, kits, and methods are disclosed for differentiating between different types of MS. Embodiments may involve detecting and/or measuring one or more diagnostic biomarkers for MS, which may include one or more IgG antibodies present within the blood serum or plasma of a subject.

After obtaining a blood serum and/or plasma sample from a subject, embodiments may involve processing the sample by exposing it to a capture composition, substrate, or matrix, which may comprise or be coated with Protein A. Protein G, or a combination of Protein A and G. The unbound eluent or flow-through collected after protein binding can then be assayed for the presence and level of one or more IgGs. IgG levels in the flow-through may be greater for subjects having MS relative to subjects not afflicted with the condition. Accordingly, IgG levels present in processed serum and/or plasma samples may be used to diagnose MS and in some examples, distinguish between MS subtypes. As used herein, a “processed” serum sample includes a serum sample that has exposed to a protein capture composition, such as a Protein A and/or G matrix. Accordingly, a processed serum sample includes the flow-through remaining after exposing an unprocessed serum sample to a Protein A and/or G matrix (or substrate or composition). A “processed” sample may also refer to a sample, e.g., a serum and/or plasma sample, that has been exposed to at least one filter, such as a 300 kDa filter. Accordingly, a processed serum and/or plasma sample may include the retentate remaining after passing an unprocessed sample through at least one filter device.

Collecting Protein A and/or G flow-through and measuring IgG levels therein was inspired in part by the discovery disclosed herein that serum IgG antibodies have not been reliably detected in blood from MS patients because the IgG antibodies often form aggregates/complexes (e.g., approximately equal to or greater than about 300 kDa), which often do not substantially bind Protein A or Protein G, both of which do typically bind IgGs. Accordingly, elevated IgG levels indicative of MS may only be detected in the unbound eluent or flow-through remaining after Protein A and/or G binding, which may be further enriched via filtration (e.g., 300 kDa filtration).

Embodiments disclosed herein utilize the discovery of IgG aggregation by providing serum-based IgG assays that involve collecting and analyzing the flow-through remaining after Protein A and/or G binding of unprocessed serum samples. Embodiments may additionally or alternatively utilize plasma and/or serum samples obtained from a subject to determine the levels of one or more IgGs (e.g., total IgG and/or IgG1) present in the retentates thereof after filtering the samples, for example with a 300 kDa or 0.1 μm column. In addition to enabling sensitive early detection of MS, the disclosed assays are rapid, non-invasive, efficient, cost-effective, quantifiable, and require small blood samples. Serum- and/or plasma-derived IgG1. IgG3, and/or total IgG levels may be used as biomarkers for MS, reaching or exceeding about 90% sensitivity and specificity. Disclosed assays may provide point-of-care diagnostic platforms, may be readily used in clinical lab settings, and may be scaled for high-throughput approaches performed at reference labs.

In certain embodiments, one or more serum and/or plasma samples obtained from a subject can be used instead of spinal fluid to diagnose MS and/or an MS subtype, in direct contrast to preexisting approaches. In accordance with these embodiments, one or more serum and/or plasma samples can be obtained from a subject having or suspected of having or developing MS and processed for additional analysis. In some embodiments, serum and/or plasma samples can be analyzed for the presence and/or the concentration of IgGs (IgG antibodies) after processing, which may involve Protein A and/or G binding, and/or one or more filtrations. It is understood by those of skill in the art that directly assaying serum samples for IgG antibodies which are known to correlate with MS has been ineffective for diagnosing MS, MS progression and MS subtypes. No difference in IgG levels was previously observed between healthy control subjects and subjects having MS. As a result, conventional practice has involved obtaining cerebral spinal fluid samples, which is painful, difficult, risky and expensive to perform.

Embodiments of the instant disclosure provide rapid and efficient assays of serum and/or plasma samples having improved accuracy, reduced discomfort for the subject, and reduced cost for diagnosing MS and MS subtypes. In some embodiments, methods disclosed herein alleviate the need to obtain and test spinal fluid from a subject. In other embodiments, methods disclosed herein are capable of distinguishing MS subtypes in order to provide improved and reliable treatment plans to a subject having a particular subtype, unlike current diagnostic methods and treatments.

In certain embodiments, serum and/or plasma samples obtained from a subject can be modest in volume and can include venous extractions of blood samples and/or a figure prick samples (e.g., about 20 to 150 μl or about 50 μl). Initial blood sample volumes required to implement one or more embodiments disclosed herein may vary, ranging from less than about 20 μl to about 20 μl, 30 μl, 40 μl, 50 μl, 60 μl, 70 μl, 80 μl, 90 μl, 100 μl, 110 μl, 120 μl, 130 μl, 140 μl, 150 μl, or more.

After obtaining the blood sample from a subject, the duration of the assays disclosed herein may be about one day or less in some examples, measured from the time of sample collection to the time the targeted IgG levels are determined and a diagnosis made. Additional examples may take longer than a day, for example up to two, three, four, five days, or more.

In some embodiments, serum and/or plasma samples can be obtained from a subject having or suspected of developing or developing MS. In certain embodiments, samples are evaluated for the presence and/or concentration of IgG antibodies and/or total protein. In accordance with these embodiments, samples can be analyzed for the presence and/or concentration of IgG antibodies in one or more serum and/or plasma sample(s) having undergone exposure to a capture composition, where the flow-through of the capture composition can be analyzed for the presence and/or amount of IgG antibodies. In some methods, total IgG antibody levels are measured and compared by methods disclosed herein. Examples may further involve enriching IgG levels by size exclusion to maximize assay sensitivity. Such embodiments may feature an enrichment device that combines multiple (e.g., two) enrichment steps. A novel spin column device for single-step sample enrichment may be configured to provide a ready-to-use clinical sample for an IgG in vitro diagnostic. The enrichment device may include at least one filter component sized to capture retentates of at least about 300 kDa.

In some examples, a plasma sample may be obtained from a subject, diluted (e.g., in PBS), filtered (e.g., using 0.1 μm microfilter tube or column), and the retentate and filtrate collected. Total protein concentrations in the retentates may be determined and compared to numerical benchmarks or total protein concentrations derived from healthy control subjects. Retentates obtained from the plasma samples derived from subjects having MS may have significantly higher protein concentrations than the retentates obtained from the plasma samples derived from healthy control subjects. Accordingly, diagnosing MS in a subject may involve collecting a plasma sample therefrom and determining a protein concentration therein, after filtering the sample, for instance using a 0.1 μm filter.

In certain embodiments, one or more of IgG1 and/or IgG3 antibody levels can be analyzed in the flow-through of the capture composition in order to diagnose MS and/or an MS subtype in a subject. The flow-through may contain or be mixed with one or more diluents, which may include PBS, TBS, and/or H₂O, at least one of which may lack or substantially lack magnesium, calcium, or both. In some embodiments, serum samples can be exposed to one or more of a Protein A and/or Protein G matrix to permit interaction with this agent. In accordance with these embodiments, non-binding serum components can be collected and analyzed to determine the level of total IgG, IgG1 and/or IgG3 antibodies or concentrations thereof in the processed sample. Processed serum samples may thus contain unnaturally high or enriched concentrations of at least one IgG compared to natural, unprocessed serum samples. In embodiments, flow-through or non-binding components can be analyzed by any method known in the art to capture IgG antibodies. In some embodiments. ELISA assays or other comparable assays, e.g., flow cytometry or Nephelometry assays, can be used to measure the presence and/or concentration or comparative levels of one or more IgG antibodies in one or more processed serum samples (e.g., after Protein A/G exposure and/or filtration). In certain embodiments, total IgG, IgG1 and/or IgG3 levels in the flow-through of a serum sample obtained from a subject having MS or an MS subtype can be about 1.5, or about 2.0, or about 2.5, or about 3.0 times greater than the level of total IgG. IgG1 and/or IgG3 in the flow-through of a control serum sample, such as a control sample obtained from a healthy subject or a subject having another neurological disorder processed by methods disclosed herein. Some embodiments may involve diagnosing a neurological disorder (e.g., MS) in a subject based on a total IgG (H+L)/Fc level, an IgG1 level, or an IgG3 level in the flow-through of the one or more serum samples compared to one or more samples obtained from a control subject not having a neurological disorder. According to such embodiments, measuring IgG (H+L)/Fc levels may involve measuring total IgG (H+L)/Fc and comparing total IgG (H+L)/Fc levels to healthy control subject samples, where elevated total IgG (H+L)/Fc levels compared to the healthy control subject samples may be indicative a neurological disorder in the subject. One or more custom ELISA protocols may be implemented in some embodiments to determine relative levels of IgG, IgG1, and/or IgG3, as further set forth below.

In some embodiments, assays disclosed herein can be used to screen one or more serum and/or plasma samples of a subject to distinguish MS subtypes. In accordance with these embodiments, methods may involve one or more of collecting one or more serum and/or plasma samples can be collected from a subject, exposing to a protein capture composition, e.g., Protein A and/or G matrix, filtering the sample(s), and/or analyzing for the presence and/or concentration of IgG antibodies (e.g. IgG1 or IgG3) in the resulting flow-through and/or retentate(s) in order to distinguish whether the subject has RRMS (Relapse Remitting MS), SPMS (Secondary Progressive MS), or PPMS (Primary Progressive MS). In some examples, a subject may be diagnosed with SPMS by determining the level of IgG1 in a processed serum sample, where the IgG1 level is elevated relative to the processed serum sample of a healthy control subject. The elevated IgG1 level in the flow-through of a subject having SPMS may be about 1.5, about 2.0, about 2.5, or about 3.0 times greater than the level of IgG1 present in a processed serum sample obtained from a healthy control subject or an unprocessed serum sample obtained from a subject having SPMS. In some examples, distinguishing between MS subtypes may involve collecting the flow-through obtained after exposing a serum sample to a Protein-A matrix or coated plate, and subsequently conducting an ELISA procedure using the flow-through according to the methods disclosed herein. Processed flow-through of serum derived from a subject having SPMS may have significantly higher levels of IgG1 than the processed flow-through obtained from subjects having PPMS or RRMS. Similarly, significantly higher levels of IgG3 may be present in processed serum samples obtained from subjects having SPMS relative to subjects having RRMS or PPMS.

In some embodiments, a subject can be diagnosed with a particular MS subtype and then treated with a treatment agent or combination of treatment agents, or not treated at all, based on the subtype. In accordance with these embodiments, a subject having SPMS may not respond to an agent being used to treat RRMS. Treatments may thus be initiated or modified based on the determined MS subtype. Reducing the level of one or more IgGs, such as IgG1, in the processed serum of a subject may cause or be indicative of effective treatment.

In other embodiments, serum and/or plasma samples and serum and/or plasma sample analysis disclosed herein can be used to assess MS progression and/or efficacy of a treatment where IgG presence and/or concentration (e.g., IgG1 and IgG3) in one or more processed serum and/or plasma samples can be measured against control samples from a subject having a particular MS subtype and non-MS or healthy controls. Cytotoxic analysis may also be performed using one or more processed serum samples to identify and optionally measure apoptosis induction. According to such embodiments, which may involve subjecting cultured human cells to the Protein-A flow-through obtained from serum samples, an apoptosis assay may be performed to determine and compare apoptosis levels in cells derived from healthy control subjects and subjects having MS, including specific MS subtypes. Increased apoptosis in processed samples relative to healthy control samples may be observed, which may be indicative of MS and/or MS progression. Notably, processed serum samples obtained from subjects having RRMS and SPMS may cause a significantly greater level of apoptotic cell death in cultured cells compared to serum derived from healthy control subjects. Serum derived from SPMS samples may also exhibit a greater cell killing capacity compared to RRMS. Accordingly, MS and one or more MS subtypes may be diagnosed by observing and/or quantifying relative apoptosis levels caused by processed serum samples (e.g., the flow-through of serum samples exposed to Protein A/G).

In certain embodiments, methods disclosed herein can be used to assess efficacy as a screening process for new or experimental agents to treat MS by processing and analyzing serum and/or plasma samples using compositions and methods of this disclosure from treated and untreated MS patients over a predetermined period. In some embodiments, reduced IgG antibodies in processed serum samples can be indicative that the treatment is reducing the side effects of MS. In accordance with these embodiments, reduced levels of IgG1 in processed serum samples disclosed herein after treatment with a target agent can be indicative of improvement and efficacy of the target agent to treat MS in the subject. It is contemplated herein that methods disclosed can be used as a platform to test MS or other neurodegenerative disorder targeting agents in order to correlate levels of IgG antibodies before, during and after treatment as a measure of improvement. In some examples. Protein A and/or G flow-through derived from plasma samples of MS patients treated with disease modifying therapies, e.g., Rituximab or Ocrelizumab, for a treatment period, e.g., about one year, may have reduced levels of IgG1 and may exhibit significantly less neuronal apoptosis.

In certain embodiments, one or more serum and/or plasma samples from a subject having, suspected of having or developing MS or other neurodegenerative disorder can be exposed one or more times to a Protein A or Protein G matrix where non-binding eluent or flow-through is collected and analyzed for levels of IgG antibodies (e.g. IgG1 and IgG3). In some embodiments, the samples can be repeatedly exposed to a Protein A or Protein G matrix, where the flow-through can be collected and applied to the same matrix or a different or unbound matrix. Sequential exposure to a protein capture composition may further enrich and/or purify the resulting processed samples, thereby increasing the accuracy of the subsequent diagnosis.

In certain embodiments, flow-through samples can be further analyzed by mass spectrometry, where protein expression levels can be evaluated to assess different subtypes of MS. Such embodiments may involve identifying clusters of differentially expressed proteins, which may coincide with MS and/or one or more MS subtypes. In some examples, for instance, processed serum samples obtained from RRPS, SPMS, and healthy control subjects may have distinct clusters of proteins capable of being separated by their relative expression levels. The expression levels of a variety of proteins may be analyzed, via Western blot or other suitable protein expression assays, to determine whether a subject has MS or a particular subtype of MS. For example, processed samples may be analyzed for the expression of IGKV1-5 (Immunoglobulin Kappa Variable 1-5); IGLV2-18 (Immunoglobulin Lambda Variable 2-18); C5 (Complement component 5); CFI (Complement Factor I); ORM1 (Orosomucoid 1); IGHV1-18 (Immunoglobulin Heavy Variable 1-18); IGHV3-49 (Immunoglobulin Heavy Variable 3-49); IGLV3-21 (Immunoglobulin Lambda Variable 3-21); LGALS3BP (Galectin 3 Binding Protein); PROC (Protein C, Inactivator Of Coagulation Factors Va And Villa); and SERPINAS (serine proteinase inhibitor), one or more of which may be significantly differentially expressed in the processed serum samples obtained from subjects having MS relative to subjects not having MS, and in subjects having RRPS relative to subjects having SPMS. Methods of distinguishing between RRPS and SPMS may thus involve detecting the expression level of certain proteins remaining in serum-derived Protein A flow-through samples, non-limiting examples of which may include IGKV1-5, IGLV2-18, C5, CFI, ORM1, IGHV1-18, IGHV3-49, IGLV3-21, PROC, or SERPINAS.

In some embodiments, flow-through samples can be analyzed by ELISA or other binding assays to rapidly examine levels of IgG antibodies in the subject processed samples against controls. In certain embodiments, control samples can be positive and/or negative controls. In other embodiments, control samples are healthy control samples. In some embodiments, samples can be compared to samples from a subject having another neurodegenerative disorder. In certain embodiments, a subject having another neurodegenerative disorder can include, but is not limited to: non-inflammatory neurological disease (NIC), inflammatory CNS disorders (IC) (9 paired with CSF), headaches such as migraine headaches due to inflammation, acute viral meningitis, B cell lymphoma, Behcet's disease, paraneoplastic syndrome, viral meningitis, chronic meningitis of unknown etiology, subacute sclerosing panencephalitis, acute disseminated encephalomyelitis, paraneoplastic encephalitis, neurosyphilis, chronic progressive meningoencephalitis, sarcoid, VZV myelopathy, VZV radiculomyelitis, VZV shingles, ischemic optic neuritis, retrobulbar optic neuritis, papillitis, and transverse myelitis Cryptococcal meningitis, neurosyphilis, sarcoid, or the like. In other embodiments for non-inflammatory CNS disorders contemplated herein, these conditions include, but are not limited to, glioblastoma, meninioma, other types of headaches, or other neurological disorder. In certain embodiments, serum samples processed and analyzed by methods disclosed herein can distinguish subjects having MS from a subject having interstitial cystitis (IC) or other neurological disorders disclosed herein or from healthy subjects, based on IgG analysis of processed serum samples described herein.

### Kits

In various embodiments, kits are contemplated for transport, storage and use of serum and/or plasma samples and/or assays disclosed herein to diagnose MS and/or distinguish an MS subtype. Kits may include Protein A and/or Protein G matrices and/or filter components configured to capture IgG aggregates that elude Protein A/G binding. Such matrix and/or filter components may have a molecular weight cut-off of about 110 kDa, 200 kDa, or 300 kDa, or may otherwise be configured to capture retentates comprising particles greater than about 200 to about 400 kDa, greater than about 250 to 350 kDa, or greater than 300 kDa. Kits may include a filtration device featuring a 0.1 μm filter, e.g., a 0.1 μm column, or a filter of comparable pore size. Associated instructions for use may also be included. In some embodiments, kits can include additional agents for treating MS in a subject or for testing a target agent for efficacy.

Protein A or Protein G matrices or columns can be used for the purification of antibodies from complex mixtures such as serum, plasma, ascites, and hybridoma culture media. Several Protein A and Protein G column and cartridge formats may be used. Protein A and G cartridges are offered with two types of Protein A or G media, and kits are available containing Protein A and Protein G columns, buffers, and desalting columns for a complete antibody purification workflow solution. Protein A beads having a high capacity of Protein A beads makes them suitable for large scale use. Protein A media can be used in biopharmaceutical production and related industries due to the high avidity of Protein A for human IgGs.

Magnet Protein A or agarose of Protein A beads for affinity chromatography can be used in methods disclosed herein, as can Protein A coated plates for collection of flow-through. The number of plates may vary, ranging from one plate to two plates, three plates, four plates, five plates or more, depending in part on the number of samples to be processed and/or the number of binding steps and flow-through collections implemented. Polyacrylamide and other polymers may also be used, in addition to or instead of Protein A agarose beads. Protein A is coupled to cross-linked agarose beads via chemically stable amide bonds. This media type is also sometimes generically called Sepharose, although this refers to a specific brand of cross-linked agarose beads. In addition to having a high capacity for antibodies, Protein A beads are very resistant to denaturing agents such as urea, chaotropic agents such as potassium thiocyanate and guanidine hydrochloride, and a wide pH range, from 2 to 11. Therefore, during harsh elution conditions that are often required to remove bound antibodies, the Protein A column may not be damaged. In certain embodiments disclosed herein, a principle difference lies in collecting and analyzing the flow-through after binding of Protein A, in contrast to common practices of purifying IgG antibodies by eluting bound IgG in Protein A and Protein G with low pH buffer. In some embodiments, acceptable bead matrices include, but are not limited to, agarose. Sepharose, acrylamide, and magnetic beads. It is noted herein that Protein A and Protein G are highly stable.

In certain embodiments, kits are provided for storage, transport and use in treating or alleviating a target disease, such an MS. In some embodiments, kits can include one or more containers. In some embodiments, kits disclosed herein contain at least one Protein A or Protein G matrix, which may comprise one or more substrates coated with Protein A or Protein G. The substrate(s) may comprise a multi-well plate, such as a 96-well plate. The substrate may also comprise a spin column or tube, e.g., microcentrifuge tube. In certain embodiments, kits disclosed herein can be portable for ease of use in remote areas for rapid analysis of serum and/or plasma samples.

In some embodiments, kits can include instructions for use in accordance with any of the methods described herein. In some embodiments, instructions can be included and can contain a description of obtaining serum and/or plasma samples from a subject. Instructions may also contain a description of processing a serum sample using Protein A and/or Protein G matrices, and/or processing the serum and/or plasma samples with a filtration device, e.g., a filtration column. In other embodiments, kits can further include a description of selecting an individual suitable for treatment based on identifying whether that individual has or is suspected of developing MS or for subtyping MS, e.g., applying the diagnostic method as described herein. In still other embodiments, the instructions can have a description of diagnosing and subtyping MS from analysis of one or more serum samples obtained. In yet other embodiments, kits can include a finger prick device or syringe or other serum and/or plasma collecting device for obtaining serum and/or plasma from a subject and containers such as tubes or other container for storing the samples prior to analysis.

Kits may feature devices, reagents, and instructions for performing a custom ELISA protocol using plasma samples or serum-derived Protein A flow-through and/or Protein G flow-through samples. Embodiments of a custom ELISA protocol may generally involve plate coating, blocking, sampling, a first washing, nutriavidin-HRP, a second washing, a TMB reaction, and a plate reading. Specific embodiments may involve coating a plate with Protein A or Protein G. The plate may be rinsed with TBST (e.g., 1×TBS diluted from 10×TBS).

In some embodiments for detecting total IgG levels, ELISA plates included with a kit may be coated with goat anti-human IgG (H+L) antibodies at a concentration of exactly or about 50 μg/ml in 0.1 M NaHCO₃, pH9.4, at 4° C. overnight. In accordance with instructions provided with the kit, for instance, the plates may be blocked with 3% BSA for about 4 hours. Plasma or flow-through samples (total 1:10.000 dilution in TBS) obtained from one or more subjects may be incubated overnight at 4° C. followed by detection with biotinylated goat anti-human IgG-Fc antibody (1:3,000 dilution) and NeutrAvidin-HRP (1:10,000, 1 hr). TBM substrate may be applied for exactly or about 30 minutes, followed by the addition of 0.1N HCl to stop the reaction. The plates may be read at exactly or about 450 nm by a microplate reader.

In some embodiments for detecting IgG1 levels, ELISA plates included with a kit may be coated with mouse anti-human IgG1 antibodies at a concentration of exactly or about 10 μg/ml in 0.1 M NaHCO₃, pH9.4, at 4° C. overnight. In accordance with instructions provided with the kit, for instance, the plates may be blocked with 3% BSA for 4 hours. Plasma or flow-through samples (total 1:10,000 dilution in TBS) obtained from one or more subjects may be incubated overnight at 4° C., followed by detection with biotinylated goat anti-human IgG-Fc antibody (1:3,000, 1 hr) and NeutrAvidin-HRP (1:10,000, 1 hr). TBM substrate may be applied for exactly or about 30 minutes, followed by the addition of 0.1N HCl to stop the reaction. The plates may be read at exactly or about 450 nm by a microplate reader.

In embodiments for detecting IgG3 levels, ELISA plates included with a kit may be coated with goat anti-human IgG (H+L) antibodies at a concentration of exactly or about 50 μg/ml in 0.1 M NaHCO₃, pH9.4, at 4° C. overnight. In accordance with instructions provided with the kit, the plates may be blocked with 3% BSA for 4 hours. Plasma or flow-through samples (total 1:1,000 dilution in TBS) obtained from one or more subjects may be incubated overnight at 4° C., followed by detection with biotinylated mouse anti-human IgG3 antibody (1:3,000, 1 hr) and NeutrAvidin-HRP (1:10,000, Ihr). TBM substrate may be applied for exactly or about 30 minutes, followed by the addition of 0.1N HCl to stop the reaction. The plates may be read at exactly or about 450 nm by a microplate reader.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the disclosure.

### Example 1

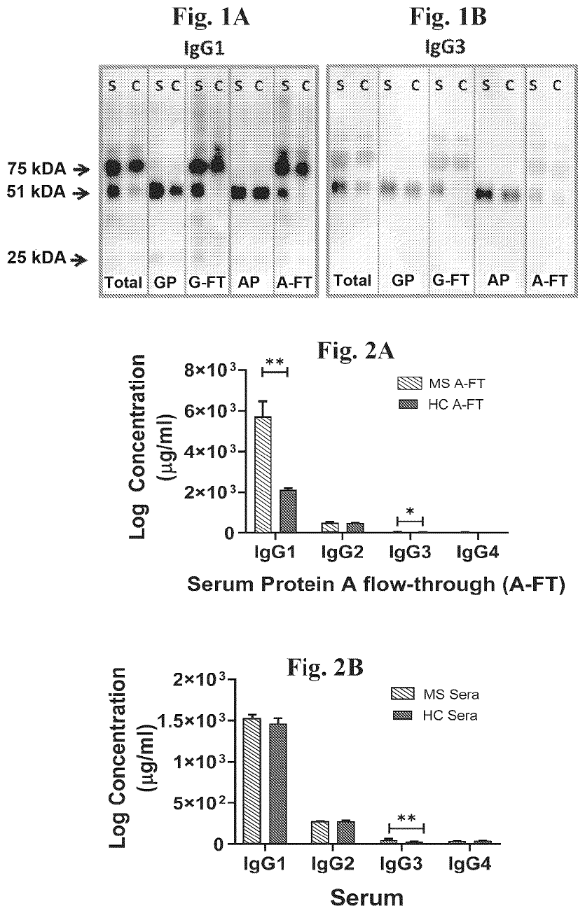

In one exemplary method, the concentrations of total glycoprotein IgG (H+L), IgG subclass IgG1, and IgG subclass IgG3 were assessed in serum and cerebrospinal fluid (CSF) collected from patients diagnosed with multiple sclerosis (MS). In brief, the MS serum and MS CSF were diluted in buffer (e.g., calcium and magnesium-free PBS, water, TBS or other suitable buffer) and absorbed through a series of Protein A- or Protein G-coated plates (e.g. Thermo) according to the methods disclosed herein. The resulting flow-through from the Protein A-coated plates was termed “A-FT” and resulting flow-through from the Protein G-coated plates was termed “G-FT”. The remaining plate-bound proteins were purified IgG proteins. Purified IgG proteins were released from the Protein A-coated plate or the Protein G-coated plate with, for example, IgG Elution Buffer and termed “AP” or “GP”, respectively. The original sample (“Total”). A-FT. G-FT, AP, and GP proteins from both serum(S) and CSF (C) were separated by SDS-PAGE gel electrophoresis followed by Western blotting with mouse anti-human IgG1 antibody (FIG. 1A) and mouse anti-human IgG3 antibody (FIG. 1B) according to the methods disclosed herein. FIGS. 1A-1B provide representative images illustrating that the 75 kDa IgG heavy chain was present in total serum/CSF. Protein A flow-through (A-FT), and Protein G flow-through (G-FT), but not in Protein A-purified IgG (AP) or Protein G-purified IgG (GP). These results demonstrate higher levels of total IgG, IgG1 and IgG3 in MS serum sample A-FT than AP. Also, a unique 75 KD band was detected that only appeared in the A-FT or G-FT, but not in the AP or GP, indicating that this larger fraction of IgG proteins were not bound to the Protein A- or Protein G-coated plate. Accordingly, MS may be diagnosed and/or monitored by obtaining one or more serum samples from a subject, subjecting the sample(s) to Protein A binding, collecting the A-FT, and determining the level of total IgG, IgG1 and/or IgG3 present therein. Relative to healthy controls, the level of IgG, IgG1, and/or IgG3 may be elevated in the sample if the subject has MS.

### Example 2

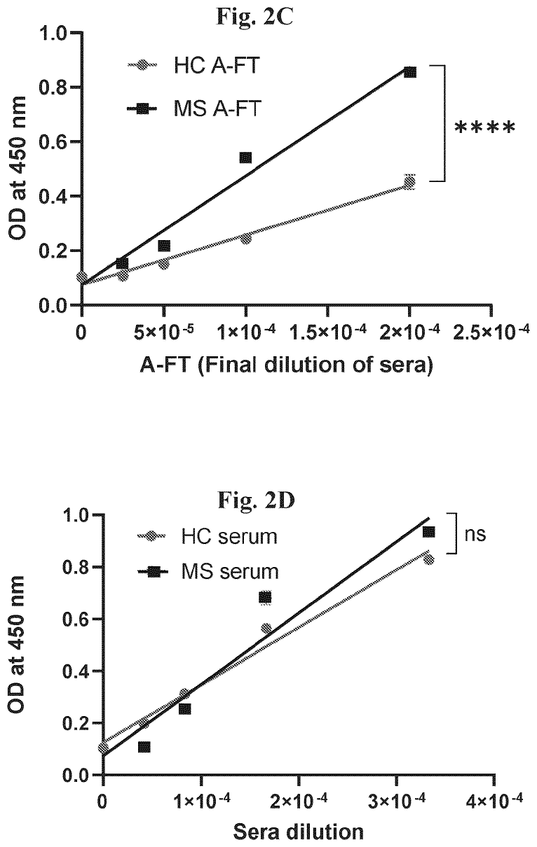

In another exemplary method, MS and healthy control (HC) serum were collected from patients, diluted at 1:100 in PBS, and then absorbed through Protein A-coated plates (any form of Protein A matrix is contemplated of use herein including, but not limited to, Protein A columns). In this exemplary method, the samples were subjected to a series of four Protein A-coated plates. It is noted that exposing a serum sample to a single Protein A-coated plate would accurately retain undesirable material while the flow-through of a single plate can be used to assess total IgG, IgG1 and/or IgG3 in the sample for diagnosing whether a subject has MS or is developing MS compared to controls. For each plate in the series of four plates, flow-through (A-FT) was collected as disclosed herein. Both MS and HC A-FT and the original serum samples were used for human IgG subclass ELISA assays, performed according to methods disclosed herein. Human IgG subclass standards were included in the ELISA assays for quantification. The results demonstrated that MS A-FT had significantly higher levels of IgG1 and IgG3 than HC A-FT (FIG. 2A). The MS sera did not have higher IgG1 than HC sera, however, there were higher levels of IgG3 in MS than HC sera (FIG. 2B). After a series of dilutions, MS A-FT consistently demonstrated significantly higher concentrations of IgG1 than HC A-FT (FIG. 2C), but MS and HC serum prior to processing demonstrated no difference in IgG1 levels (FIG. 2D). It was observed that higher IgG1 and IgG3 and in certain examples, total IgG (H+L) and total IgG Fc were only found in Protein A flow-through and not identified prior to processing, for example in unprocessed serum samples that were not exposed to Protein A and/or Protein G.

### Example 3

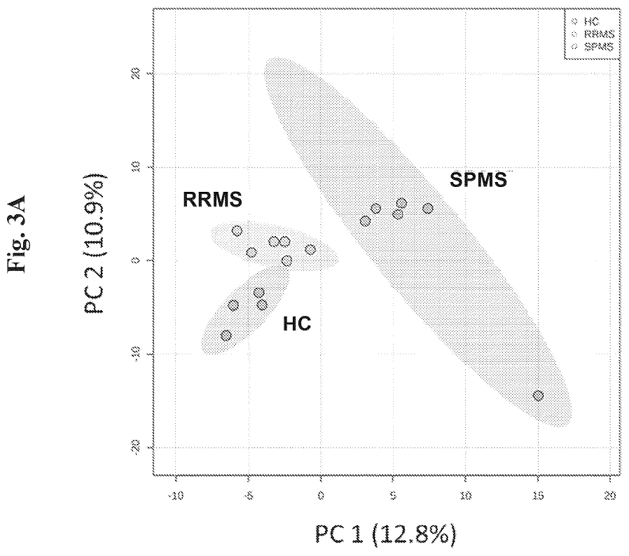

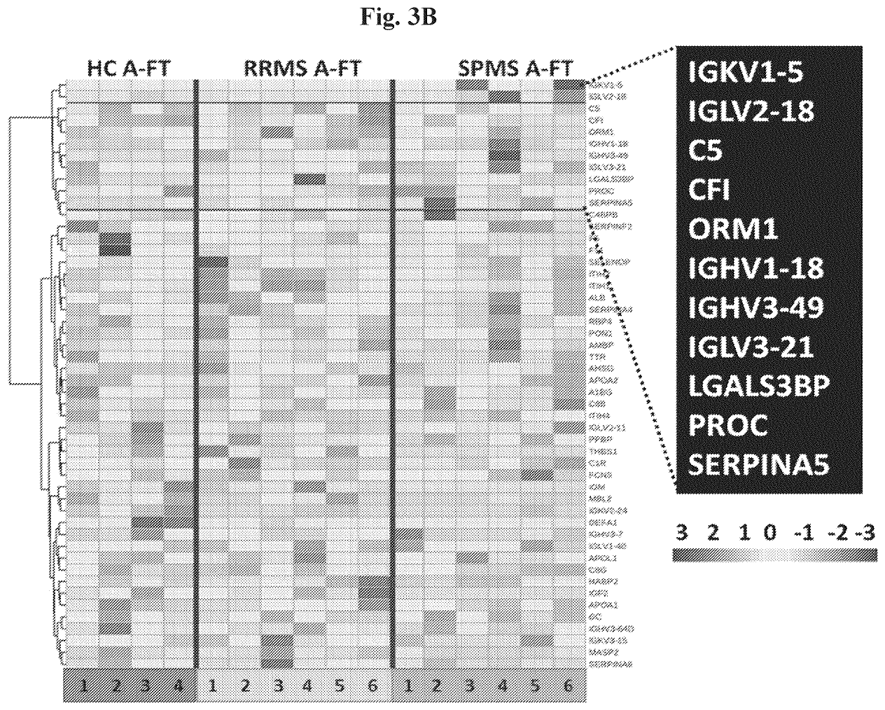

In another exemplary method, mass spectrometry analysis was performed to determine protein profiles in sera collected from patients having one of two types of MS: Relapsing-Remitting MS (RRMS) or Secondary-Progressive MS (SPMS). In brief, serum was collected from healthy control (HC), RRMS, or SPMS patients, diluted at 1:100 in PBS, and then absorbed onto Protein A-coated plates. Flow-through (A-FT) was collected as disclosed herein. The A-FT samples were then processed for mass spectrometry analysis according to methods disclosed herein. The proteins identified from each group of samples were graphed in FIG. 3A, in which PC represents the Principle Component of each sample. The data demonstrated that RRPS, SPMS, and HC each had distinct clusters of proteins capable of being separated by their relative expression levels. The protein heat map also demonstrated that many unique immunoglobulins and complement components were found at the highest levels in SPMS A-FT, modest levels in RRMS-A-FT, and lowest in HC A-FT (FIG. 3B). As illustrated in FIG. 3B, the following IgG and complements were significantly differentially expressed in MS A-FT compared to HC A-FT: IGKV1-5 (Immunoglobulin Kappa Variable 1-5); IGLV2-18 (Immunoglobulin Lambda Variable 2-18); C5 (Complement component 5); CFI (Complement Factor I); ORM1 (Orosomucoid 1); IGHV1-18 (Immunoglobulin Heavy Variable 1-18); IGHV3-49 (Immunoglobulin Heavy Variable 3-49); IGLV3-21 (Immunoglobulin Lambda Variable 3-21); LGALS3BP (Galectin 3 Binding Protein); PROC (Protein C, Inactivator Of Coagulation Factors Va And VIIIa); and SERPINAS (serine proteinase inhibitor). Disclosed methods of distinguishing between RRPS and SPMS may thus involve detecting the expression level of certain proteins remaining in serum-derived A-FT samples, non-limiting examples of which may include IGKV1-5, IGLV2-18, C5, CFI, ORM1, IGHV1-18, IGHV3-49, IGLV3-21, PROC, or SERPINAS.

### Example 4

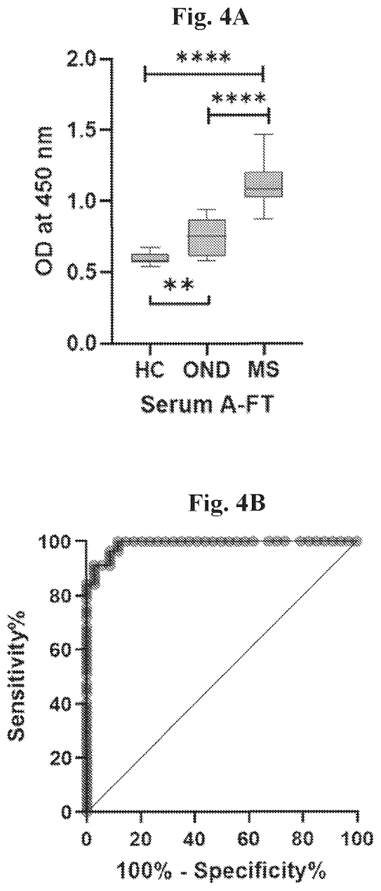

In another exemplary method, IgG1 was assessed in MS serum as a disease marker. In brief, an adapted ELISA procedure was developed to measure IgG1 levels from serum samples according to the methods disclosed herein. Serum was collected from healthy control (HC) patients, MS patients, and patients diagnosed as having other neurological diseases (OND), diluted at 1:100 in PBS, and then absorbed through Protein A-coated plates. Flow-through (A-FT) was collected as disclosed herein. A-FTs were then subjected to adapted ELISA procedure according to the methods disclosed herein. The ELISA result demonstrated that MS A-FT had the highest levels of IgG1 relative to both HC and OND A-FTs (FIG. 4A). Specificity of ELISA IgG1 signal of MS A-FT was graphed against the sensitivity of ELISA IgG1 signal in all controls combined, and it was discovered that the area under the ROC (receiver operating characteristic) curve was 0.9892 (FIG. 4B), indicating that the assay was both specific and sensitive. IgG1 levels in serum A-FT may thus be analyzed to distinguish not only between healthy subjects and subjects having MS, but between subjects having MS and subjects having one or more other neurological diseases.

### Example 5

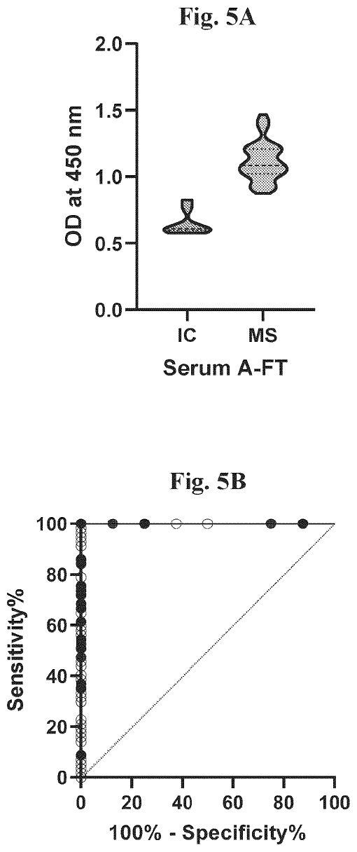

In another exemplary method, the ability to measure serum IgG concentrations to differentiate between MS and other inflammatory CNS disorders or conditions (“IC”) was assessed. In brief, serum was collected from MS patients and patients diagnosed as having various ICs, such as meningitis, diluted at 1:100 in PBS, and then absorbed through Protein A-coated plates. Flow-through (A-FT) was collected as disclosed herein. A-FTs were then subjected to adapted ELISA procedure according to the methods disclosed herein. The results demonstrated that MS serum A-FT had significantly higher levels of IgG1 than IC serum A-FT (FIG. 5A). Specificity of ELISA signal was graphed against the sensitivity of ELISA signal and it was found that these two parameters correlated, with a near-perfect ROC curve (FIG. 5B), indicating the assay was both specific and sensitive.

### Example 6

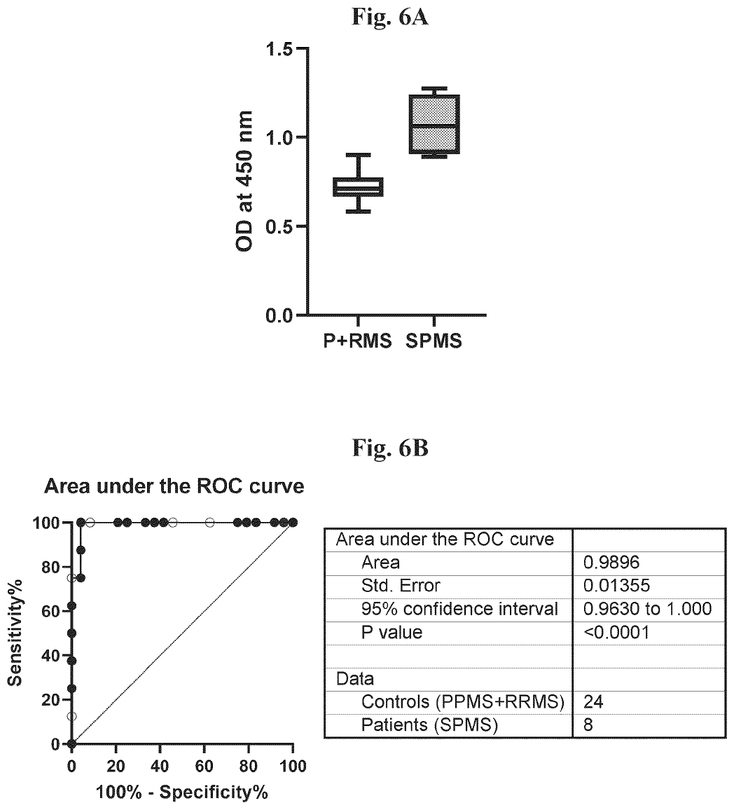

In another exemplary method, the ability of measuring concentrations of serum IgG to differentiate between MS types was assessed. In brief, serum or plasma was collected from patients having one of three types of MS: Relapsing-Remitting MS (RRMS), Secondary-Progressive MS (SPMS), or Primary-Progressive MS (PPMS). Sera was diluted at 1:100 in PBS and then absorbed through Protein A-coated plates. Flow-through (A-FT) was collected as disclosed herein and then subjected to an adapted ELISA procedure according to the methods disclosed herein. The results demonstrated that SPMS serum A-FT had significantly higher levels of IgG1 than PPMS A-FT and RRMS A-FT (FIG. 6A). Similarly, significantly higher levels of IgG3 were also detected in SPMS A-FT compared to other 2 subtypes of MS (RRMS and PPMS). This is significant, as there are currently no biomarkers able to differentiate SPMS from other types except by clinical worsening presentations and disability scores (e.g., waiting for progression to occur). Further, there are no effective therapies for SPMS. The IgG1 and IgG3 blood assays disclosed herein can assist with evaluating efficacy of experimental agents to treat SPMS by measuring IgG levels before, during and after and experimental agent is used. Specificity and sensitivity were demonstrated by AUC (area under the curve) with a near-perfect ROC curve (FIG. 6B), indicating the assay was both specific and sensitive. It is significant that both MS and IC CSF demonstrated oligoclonal bands.

### Example 7

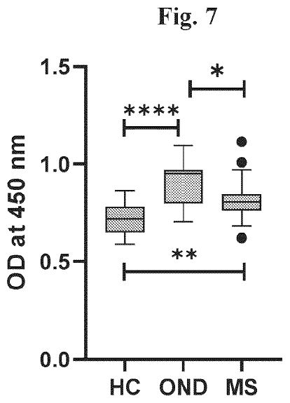

In another exemplary method, IgG levels were assessed in Protein A-bound IgG isolated from healthy control (HC) patients, MS patients, and patients diagnosed as having other neurological diseases (OND) according to the methods disclosed herein. In brief, Protein A-bound protein samples were prepared and subjected to an ELISA assay to measure IgG according to the methods disclosed herein. A goat anti-human IgG-Fc antibody was used to detect total IgG. The results demonstrated that MS serum had a significantly lower amount of IgG as compared to that from OND serum (FIG. 7, p=0.02). In addition, both OND and MS had higher IgG levels than HC. The results demonstrated different neurological diseases had different IgG levels and distinctive Protein A binding patterns.

### Example 8

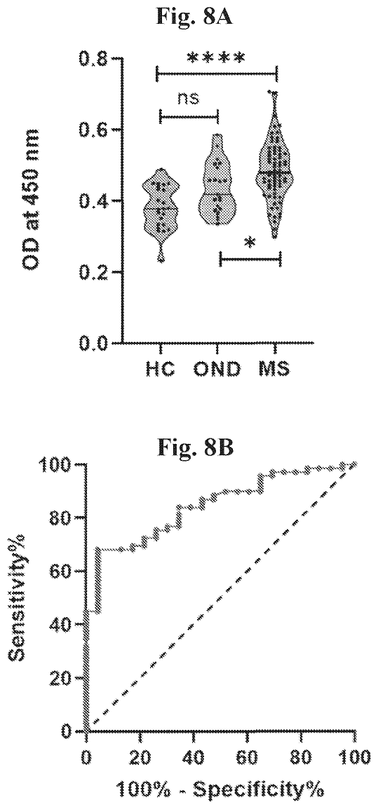

In another exemplary method, IgG3 levels in sera collected from isolated from healthy control (HC) patients, MS patients, and patients diagnosed as having other neurological diseases (OND) were assessed. In brief, serum was collected from HC, MS, and OND patients, diluted at 1:100 in PBS, and then absorbed through Protein A-coated plates. Flow-through (A-FT) was collected and then subjected to an adapted ELISA procedure according to the methods disclosed herein. The results demonstrated that MS serum A-FT had significantly higher levels of IgG3 as compared to that from HC and OND serum A-FT samples (FIG. 8A). The specificity vs sensitivity graph demonstrated this with the area under the ROC (receiver operating characteristic) curve at 0.84 (FIG. 8B), indicating the assay was both specific and sensitive. Accordingly, the level of IgG3 measured in A-FT samples may serve as a reliable biomarker for MS, and may be used to distinguish MS from other neurological diseases.

### Example 9

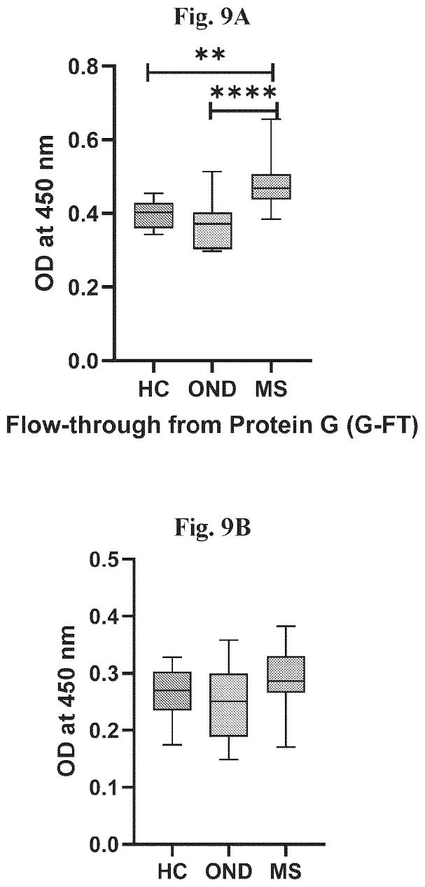

In another exemplary method, IgG1 and IgG3 levels were assessed in sera isolated from healthy control (HC) patients, MS patients, and patients diagnosed as having other neurological diseases (OND) after exposure of the sera to Protein G. In brief, serum was collected from HC, MS, and OND patients, diluted at 1:100 in PBS, and then absorbed through Protein G-coated plates. Flow-through (G-FT) was collected and then subjected to an adapted ELISA procedure according to the methods disclosed herein to analyze the IgG1 and IgG3 levels in G-FT samples. The results demonstrated that MS serum G-FT had significantly higher levels of IgG1 as compared to that from HC and OND serum G-FT samples (FIG. 9A). However, there were no significant differences in the IgG3 levels among HC, OND and MS G-FT samples (FIG. 9B), indicating that the level of IgG1, but not IgG3, remaining in G-FT samples derived from patient serum may be indicative of MS.

### Example 10

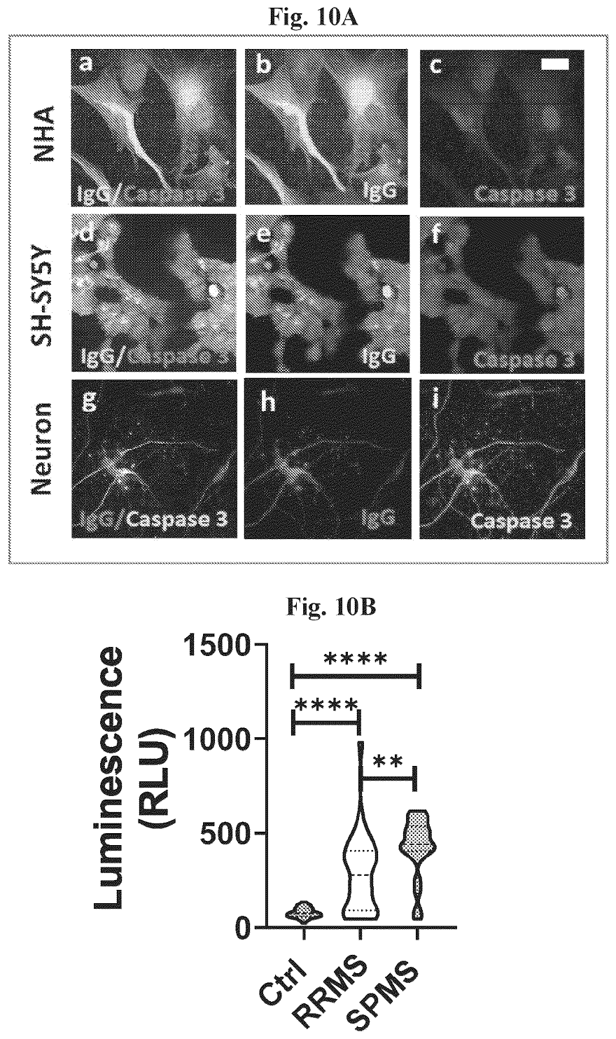

In another exemplary method, cytotoxicity of Protein A flow-through collected from sera of serum MS and HC patients was assessed in cultured human cells. In brief, cytotoxicity tests of MS and HC A-FT were performed in cultured human cells, including primary human astrocytes (NHA), neuroblastoma SH-SY5Y cells, and primary neurons according to methods disclosed herein (FIG. 10A). The MS or HC A-FT and 5% of Normal Human Serum (as a source of complements) were added to the neural culture. The cells were fixed after 2 hours for immunostaining using anti-human IgG antibody and anti-Caspase 3 antibodies (a marker of apoptosis). The confocal imaging demonstrated co-localization of both human IgG and Caspase 3 in all three cell types (FIG. 10A), indicating IgG antibody-induced apoptotic cell death. Apoptotic/necrotic cell death was also measured the using a luminescence/fluorescence plate reader after the MS A-FT or HC A-FT treatment of the cells. The results demonstrated that both RRMS and SPMS A-FT produced a significantly higher number of apoptotic cell death as compared to HC A-FT with higher cell killing capacity for SPMS compared to RRMS (FIG. 10B). The MS serum A-FT-induced cell death was only present in an A-FT preparation, but not directly from MS serum (data not shown).

### Example 11

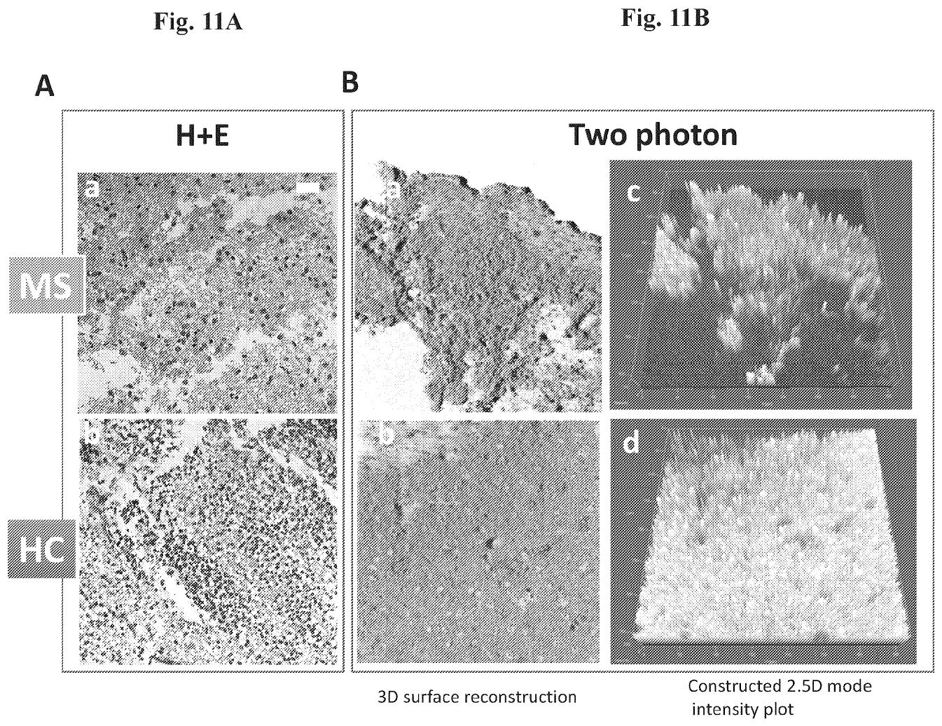

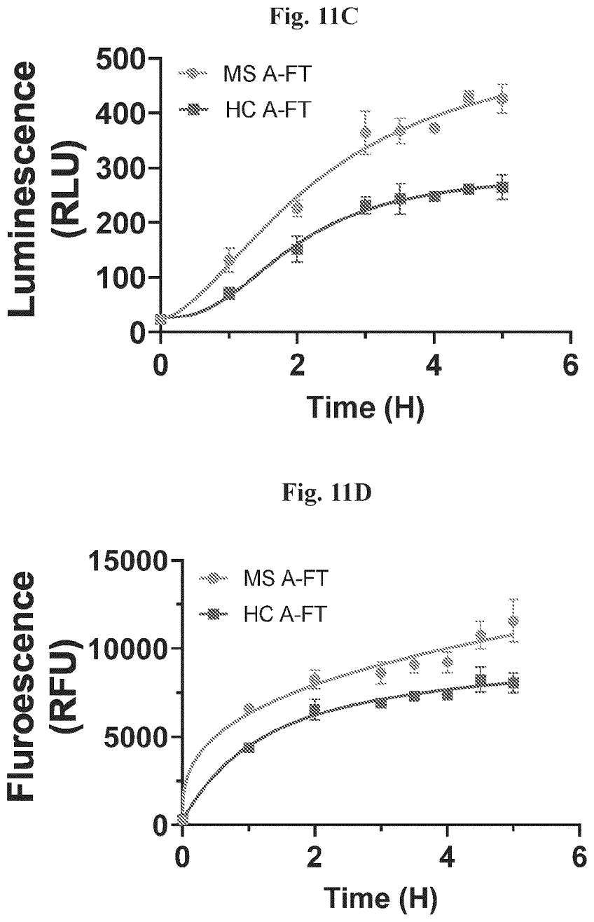

In another exemplary method, cytotoxicity of Protein A flow-through collected from sera of MS patients was assessed ex vivo. In brief, cytotoxic studies of MS serum IgG in A-FT were performed using newborn (P1) mouse cerebral tissue slices according to methods disclosed herein. The MS A-FT or HC A-FT were incubated with the tissue slices for 1-3 hours before microscopic analysis. Two-photon microscopy was used to evaluate tissue damage in unfixed slices (2 mm). Compared to control HC A-FT. MS serum IgG caused apparent brain tissue damage and cell death as early as after one hour of treatment, as illustrated in H&E staining (FIG. 11A). Progressive brain tissue damage was observed after 3 hours of incubation as reflected by 2-Photon deep imaging and 3D XYZ imaging (FIG. 11B). Similar to human neuronal cells, higher levels of apoptosis and necrosis were produced by MS A-FT as compared to HC A-FT in the brain tissues, as illustrated in the luminescence graph (FIG. 11C for apoptosis) and fluorescent graph (FIG. 11D for necrosis).

### Example 12

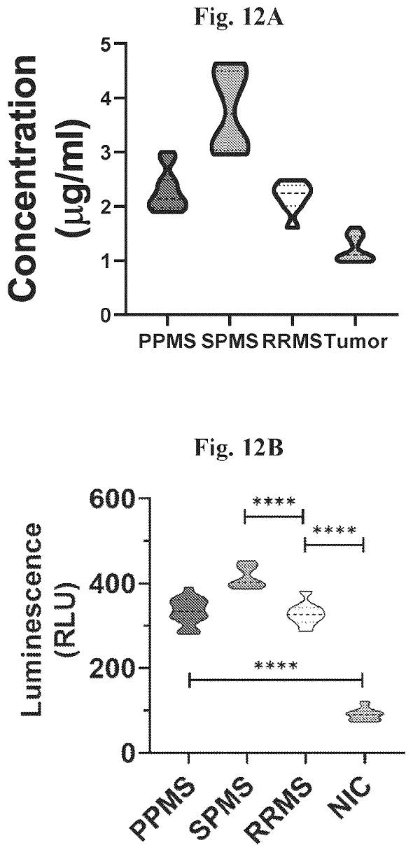

In another exemplary method, a correlation between the IgG in Protein A flow-through collected from sera of MS patients and neuron killing (apoptosis) was assessed. In brief, the A-FT sample was prepared from PPMS, SPMS, RRMS, and brain tumor serum according to the methods disclosed herein. An adapted ELISA assay was performed to determine the IgG1 levels in serum A-FT samples. Results demonstrated that serum derived from subjects having SPMS had significantly higher levels of IgG1 than that from PPMS, RRMS, and Tumor A-FT (FIG. 12A). The cytotoxicity of these A-FT samples was also tested in SH-SY5Y neuroblastoma cells. It was found that SPMS had the highest apoptosis rate, followed by PPMS and RRMS, with all three MS A-FT produced significantly higher apoptotic cell death than control NIC A-FT (FIG. 12B). The ELISA and functional cytotoxicity results demonstrated that there was a strong correlation between higher IgG1 in MS serum A-FT and their toxic effect on neurons.

### Example 13

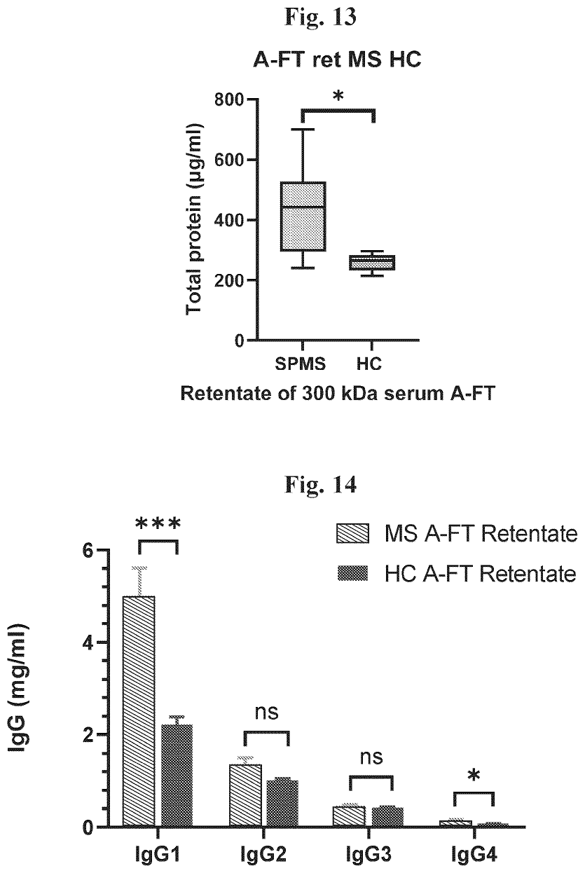

In another exemplary method, total proteins in the retentates of Protein A flow-through collected from sera of SPMS and HC patients was assessed. In brief, a 300 KDa molecular weight cut-off filter tube (Pall Life Science, Nanosep 300KD, Omega, #OD300C34) was used to separate both SPMS and HC serum A-FT proteins based on their protein size. After centrifugation, the fraction in the top sample tube (i.e., the “retentate”) was collected and stored at −80° C. The total protein concentrations in this retentate fraction was measured with the BCA method according to the methods disclosed herein. It was found that SPMS A-FT had significantly higher protein concentrations than the HC A-FT Retentate (FIG. 13). Since the A-FT starting volume and final retentate volume were the same for both SPMS and HC, the result indicated that there was a higher amount of total proteins being retained in high molecular weight fraction in SPMS serum A-FT than HC serum A-FT samples. Accordingly, methods disclosed herein for diagnosing MS and/or SPMS may involve obtaining a serum sample from a subject, exposing the sample to a Protein A substrate (e.g., matrix, plate, column, etc.), filtering the flow-through using a 300 kDa filter, collecting the retentate, and determining the total protein concentration therein. The total protein concentration can be compared to serum samples processed in the same manner. Diagnosing MS or an MS subtype, e.g., SPMS, may involve determining that a greater amount of total proteins are present in the retentate.

### Example 14

In another exemplary method, amounts of each IgG subclass were assessed in the retentates of Protein A flow-through collected from sera of SPMS and HC patients. In brief, a 300 KDa filter tube was used to separate A-FT proteins as according to the methods disclosed herein. After the centrifugation, the fraction in the top sample tube (the retentate) was collected and stored at −80° C. The human IgG subclass levels were measured in this retentate fraction using an ELISA kit (Invitrogen) following the recommended protocol. It was found that SPMS A-FT had significantly higher IgG1 and IgG4 than the HC A-FT retentate (FIG. 14). The MANOVA was less than 0.0001 between MS and HC A-FT Retentate. The IgG2 and IgG3 in the SPMS A-FT retentate were not significantly different from that in HC A-FT retentate.

### Example 15

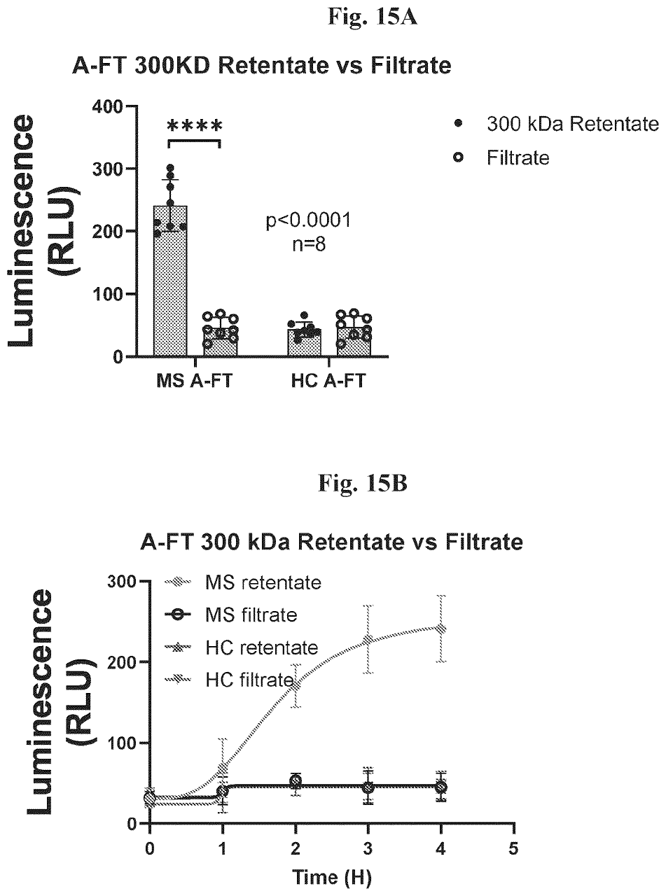

In another exemplary method, cytotoxicity was assessed for the retentates of Protein A flow-through collected from sera of MS and HC patients. In brief, a 300 KDa filter tube was used to separate A-FT proteins as described herein. Both the top retentates (high molecular weight fraction) and the bottom (lower molecular weight fraction (also referred to as “Filtrates”) were collected and stored at −80° C. for later analysis. A cytotoxicity test of retentates and filtrates was then performed in neuroblastoma SH-SY5Y cells using apoptosis-marker luminescence for measurement of cell death. It was found that only MS A-FT retentate produced significant apoptosis in SH-SY5Y cells, but not HC A-FT retentate (FIG. 15A). Neither MS nor HC A-FT filtrate generated any cytotoxicity. In a time-course graph, it was found that MS A-FT 300 KDa retentate produced time-dependent neurotoxicity (FIG. 15B).

### Example 16

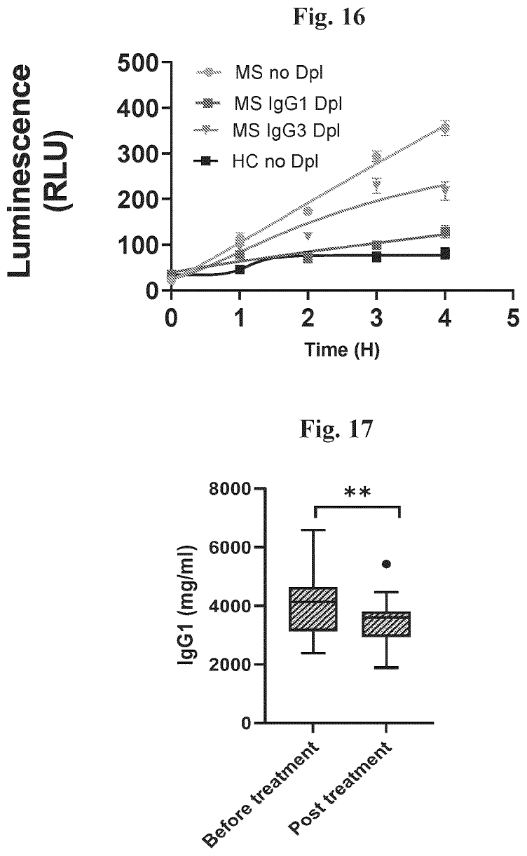

In another exemplary method, cytotoxicity tests of IgG subclass-depleted MS and HC A-FT were performed in SH-SY5Y cells. In brief, Biotin labeled IgG1 and IgG3 antibodies were incubated with A-FT, followed by binding to Strapavidin magnet beads. The flow through (IgG1 IgG3 depleted) were used to test for cytotoxicity in SH-SY5Y cells with an apoptosis luminescence marker according to the methods disclosed herein. It was found that the IgG1-depleted MS A-FT retentates were significantly less toxic to cells, while the IgG3-depleted MS A-FT retentates had partially reduced cytotoxicity (FIG. 16). The IgG2 and IgG4 antibody depleted MS A-FT retentate did not demonstrate as many differences as compared to non-depleted MS A-FT Retentate (data not shown). Accordingly. IgG1 reduction or depletion may abolish or significantly reduce neuronal toxicity. Therapies targeting IgG1 in MS patients may thus reduce or slow neuronal cell death in MS patients relative to the neuronal cell death otherwise likely to occur in the same patients.

### Example 17

In another exemplary method, levels of IgG1 in MS serum collected after treatment with disease-modifying therapies was assessed. Anti-B cell antibody therapies are the most commonly used DMT for MS. Some of the most common DMT for MS include, but are not limited to, Interferon beta 1b, Interferon beta 1a, Glatiramer acetate, Natalizumab, Fingolimod, Teriflunomide, Dimethyl fumarate. Alemtuzumab, Daclizumab. Ocrelizumab, Laquinimod and others. In brief, an adapted developed ELISA protocol disclosed herein was used to measure the IgG1 levels in MS serum A-FT that had been collected before and after the drug treatment (Pre, before treatment: V1, the first visit 3-6 months after drug treatment: V2, the second visit 9-12 months after drug treatment). The MS patients had received rituximab (RTX) and ocrelizumab (OCR) antibody infusion. The ELISA results demonstrated that the drug treatment (patients were combined for both drugs) generally led to lower IgG1 levels in MS serum A-FT as compared to pre-treatment serum A-FT (FIG. 17). Since the drug treatment generally resulted in MS symptom alleviation and motor functional improvements, the results indicated that the drug-induced positive outcome may be associated with lower serum A-FT IgG1 antibody levels.

### Example 18

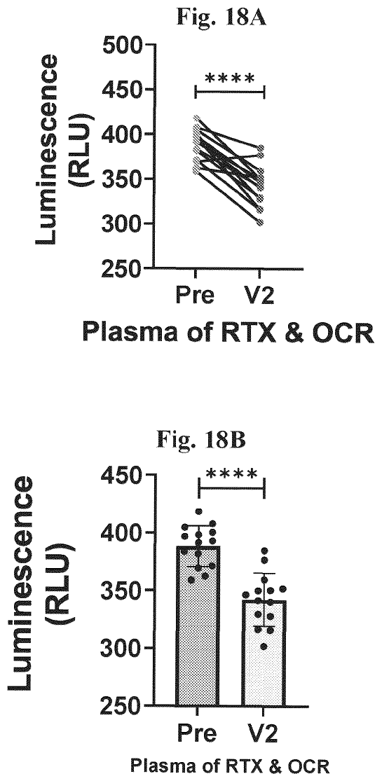

In another exemplary method, cytotoxicity of serum collected from rituximab (RTX) and ocrelizumab (OCR)-treated MS patients was assessed. In brief, MS serum A-FT samples were prepared from patients receiving rituximab (RTX) and ocrelizumab (OCR) antibody infusion treatment, including baseline serum (pre-treatment) according to methods disclosed herein. Then cytotoxicity tests were performed the in SH-SY5Y cells according to the methods disclosed herein. The apoptosis marker luminescence was used for measurement of cell death. Results demonstrated that V2 A-FT (the second visit after drug treatment) caused significantly lower apoptotic cell death as compared to pre-treatment serum A-FT (Pre) (FIG. 18A, individual samples). When combined as a group, the V2 A-FT demonstrated significantly lower apoptosis than Pre A-FT (FIG. 18B). Accordingly, serum A-FT of MS patients treated with disease-modifying therapies had reduced levels of IgG1 and produced significantly less neuronal apoptosis after treatments of either RTX or OCR.

### Example 19

In another exemplary method, an adapted ELISA assay was used to determine total IgG concentrations in MS and control A-FT samples derived from plasma. Samples were obtained from patients having RRMS (68 samples), PPMS (6 samples), and SPMS (26 samples). Inflammatory control samples (IC), healthy control samples, (HC), and non-inflammatory CNS control samples (NIC) were measured as well. Plasma samples (each 2 μL diluted with 198 μL of PBS) were added to the wells of Protein A-coated 96-well plates and incubated at 4° C. for two to five hours. The unbound solution/supernatant (A-FT) of each sample was transferred to a second well and incubated overnight at 4° C. A second volume of A-FT was then collected, and a similar procedure was used to collect a third A-FT sample. The final A-FT was aliquoted and stored at −80°. Protein G flow-through collections were also obtained using the same procedure.

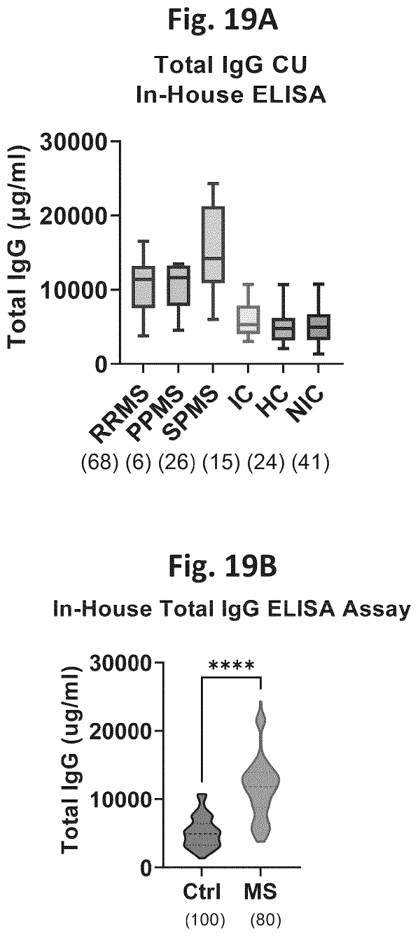

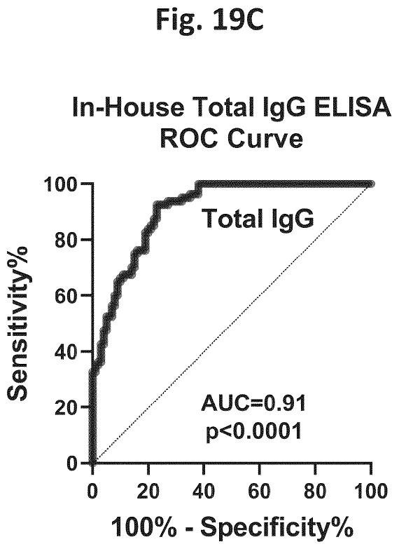

The adapted ELISA protocol was used to determine total IgG levels in MS and control A-FT samples. As shown in FIG. 19A, MS A-FT obtained from RRMS, PPMS, and SPMS patients contained higher levels of total IgG than healthy controls, with SPMS having the highest level of total IgG, reaffirming that MS A-FT can be utilized to distinguish SPMS from other MS subtypes by measuring the IgG levels therein. FIG. 19B shows that MS A-FT had significantly higher total IgG levels than control A-FT samples (****p<0.0001), and FIG. 19C shows that the ROC curve of the adapted total IgG ELISA had an area of 0.91 (p<0.001), indicating the assay was both specific and sensitive.

### Example 20

ROC curves for IgG1, IgG3, and total IgG ELISA assays were determined and analyzed for each of separate cohorts of subjects to confirm the specificity and sensitivity of the assays. The first cohort, named the “CU cohort.” included 182 total subjects, including six subjects having PPMS, 26 having SPMS, 70 having RRMS, 24 healthy controls, 15 inflammatory controls, and 41 having some other neurological disease, which included glioblastoma WHO grade IV, meningioma WHO grade I, astrycytoglioma grade III, and schwannom grade I. A second cohort, named the “ACP cohort.” included 170 total subjects, including 28 subjects having PPMS. 10 having SPMS. 52 having RRMS, 28 healthy controls, and 52 having some other neurological disease or condition, which included glioblastoma WHO grade IV, meningioma WHO grade I, astrycytoglioma grade III, Alzheimer's disease, and a traumatic brain injury. Table 1, below, shows the AUC value of MS subjects relative to all control samples (“Ctrl”), health controls (“HC”), other neurological diseases (“OND”), along with the sample sizes for each group. As evident from the AUC values, ranging from 0.83 up to 0.96, the ELISA assays accurately showed that significantly higher levels of total IgGs are detected in MS A-FT than control A-FT.

TABLE 1
Blood IgG Biomarkers for MS

| | | AUC | AUC | AUC | | | |
|---|---|---|---|---|---|---|---|
| | | [Ctrl] | [HC] | [OND] | MS (n) | | |
| | | (95% | (95% | (95% | (RRMS/ | HC | OND |
| Biomarker | Cohort | CI) | Cl) | Cl) | SPMS/PPMS) | (n) | (n) |
| IgG1 | CU | 0.87 | 0.86 | 0.88 | 100 | 24 | 56 |
| | ACP | 0.91 | 0.87 | 0.96 | 90 | 20 | 16 |
| IgG3 | CU | 0.86 | 0.89 | 0.85 | 100 | 24 | 56 |
| | ACP | 0.86 | 0.87 | 0.84 | 90 | 20 | 16 |
| Total IgG | CU | 0.91 | 0.91 | 0.90 | 100 | 24 | 56 |
| | ACP | 0.85 | 0.86 | 0.83 | 90 | 20 | 16 |

### Example 21

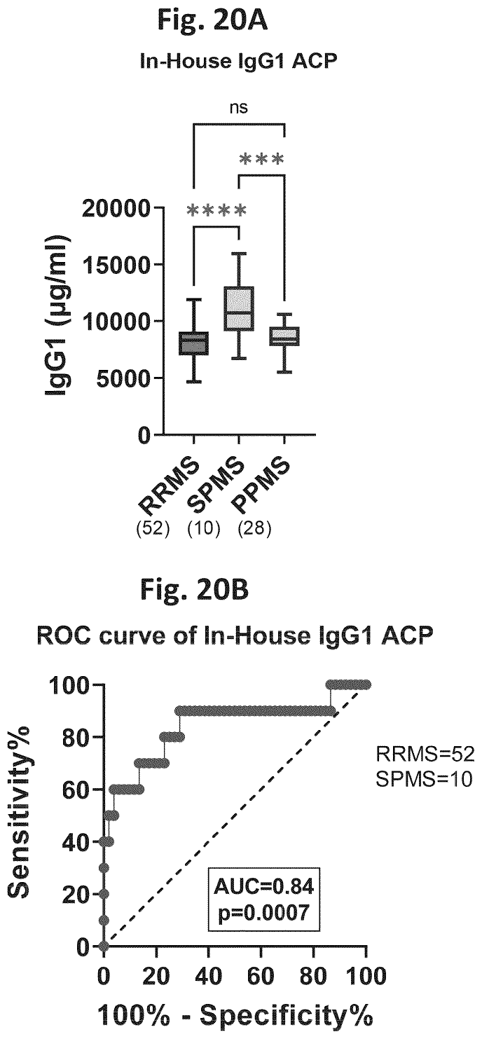

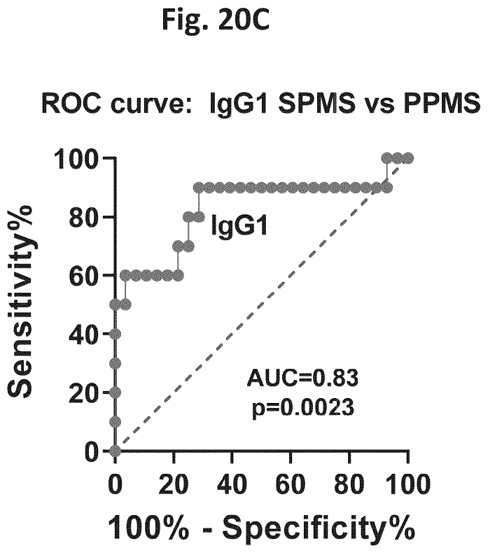

An adapted ELISA protocol was performed to determine IgG1 levels in the serum-derived A-FT of subjects having RRMS, PPMS, and SPMS. As shown in FIG. 20A, SPMS A-FT had significantly higher IgG1 levels than RRMS A-FT and PPMS A-FT (****p<0.0001), ***p<0.001). The ROC curve depicted in FIG. 20B shows that SPMS versus RRMS had an area of 0.84 (p<0.0007), and FIG. 20C shows that the ROC curve of SPMS versus PPMS had an area of 0.84 (p=0.0023). Accordingly, IgG1 levels present in serum-derived A-FT may be utilized to distinguish subjects afflicted with SPMS from subjects having RRMS and/or PPMS. The corresponding ROC curves for SPMS versus RRMS and SPMS versus PPMS are shown below in Table 2, along with the sample sizes for each group.

TABLE 2
IgG Biomarkers for SPMS

| | | AUC [RRMS] | AUC [PPMS] | SPMS | RRMS | PPMS |
|---|---|---|---|---|---|---|
| Biomarker | Cohort | (95% CI) | (95% Cl) | (n) | (n) | (n) |
| IgG1 | CU | 0.76 | 0.66 | 26 | 68 | 6 |
| | | (p < 0.0001) | (p = 0.2274) | | | |
| | ACP | 0.84 | 0.83 | 10 | 52 | 28 |
| | | (p = 0.0007) | (p = 0.0023) | | | |
| IgG3 | CU | 0.73 | 0.69 | 26 | 68 | 6 |
| | | (p = 0.0005) | (p = 0.1615) | | | |
| | ACP | 0.68 | 0.65 | 10 | 52 | 28 |
| | | (p = 0.069) | (p = 0.1638) | | | |
| Total IgG | CU | 0.72 | 0.72 | 26 | 68 | 6 |
| | | (p = 0.0009) | (p = 0.0911) | | | |
| | ACP | 0.69 | 0.64 | 10 | 52 | 28 |
| | | (p = 0.0607) | (p = 0.2078) | | | |

### Example 22

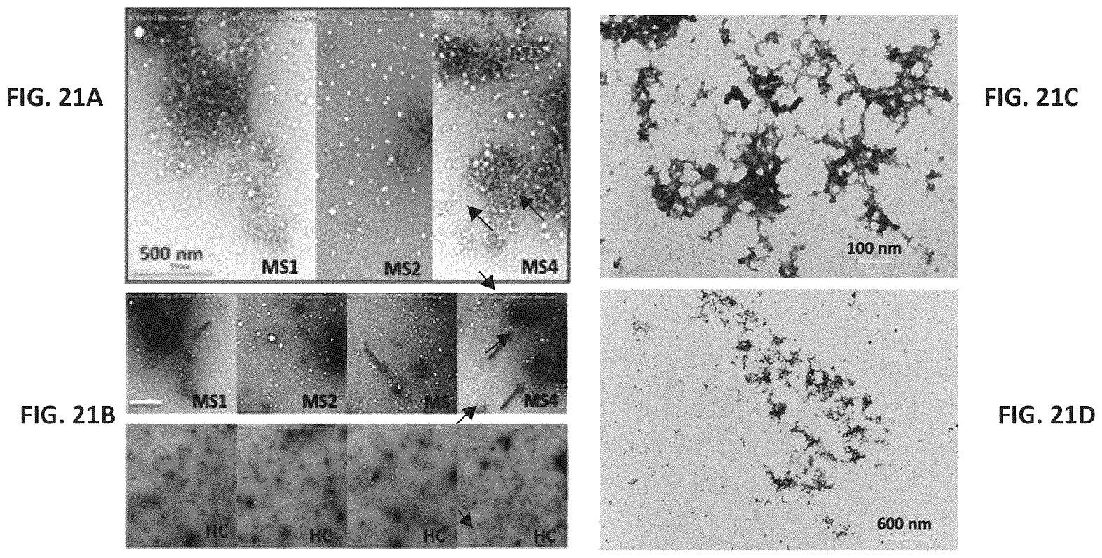

In another exemplary method, SPMS and HC A-FT derived from plasma samples were passed through a 300 kDa microtube filter, which separated proteins into larger retentates and smaller filtrates. Transmission electron microscopy (TEM) was used to obtain magnified views of the retentates and filtrates. FIG. 21A shows that IgG formed large aggregates in the 300 kDa retentates collected from three individual MS A-FT samples. FIG. 21B shows that the MS A-FT samples contained IgG aggregates (top panel, arrows), but the HC A-FT did not (lower panel). FIG. 21C shows that IgG obtained from MS A-FT retentates (stained with uranyl acetate) formed large aggregates, whereas HC A-FT retentates did not (FIG. 21D). Accordingly, A-FT samples derived from subjects having MS contain large protein aggregates that may be detected for MS diagnosis.

### Example 23

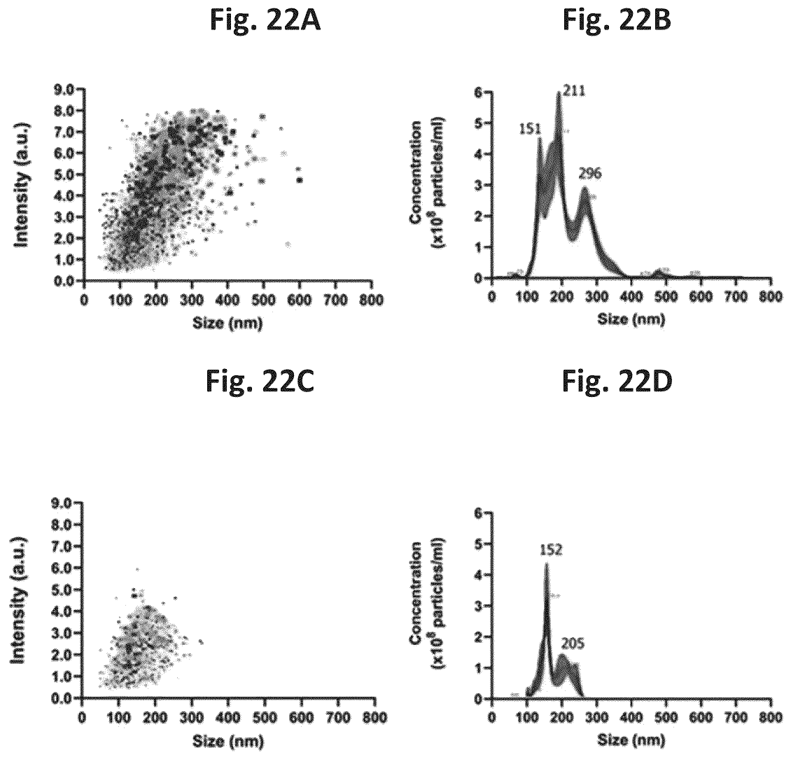

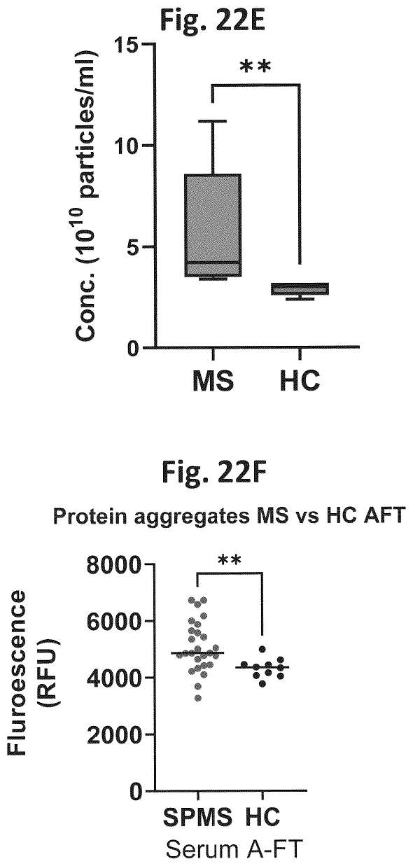

In another exemplary method, nanoparticle tracking analysis was performed to examine particle sizes contained within the aforementioned A-FT samples. As shown in FIGS. 22A, 22B, 22C, and 22D, MS A-FT 300 kDa retentates contained larger particles (FIGS. 22A and 22B; peak at 211 nm) relative to HC A-FT samples (FIGS. 22C and 22D, peak at 152 nm). Quantification of the nanoparticle tracking analysis, depicted graphically in FIG. 22E, showed that MS A-FT 300 kDa retentates had approximately twice the amount of particles (>100 nm) than HC A-FT (**p<0.01, n=4 each). A fluorescent protein aggregate reporting dye was used to detect the levels of protein aggregates in MS and HC A-FTs. The results, depicted in FIG. 22F, show that MS A-FT retentates had significantly greater numbers of protein aggregates relative to HC A-FT retentates (**p<0.01).

### Example 24

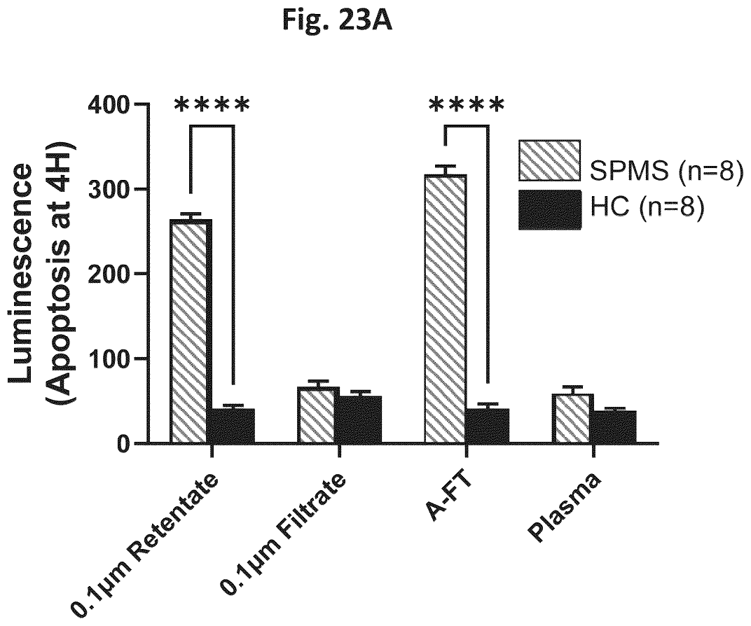

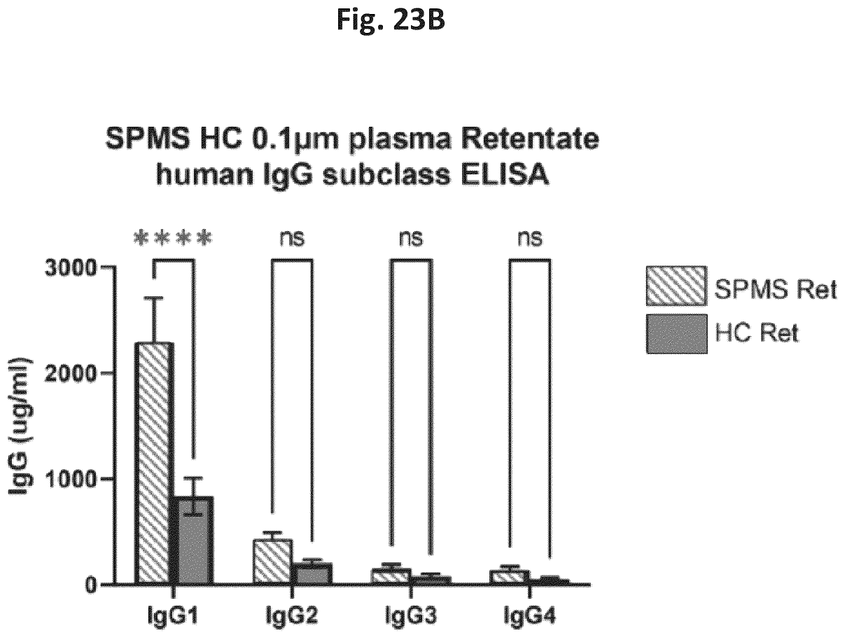

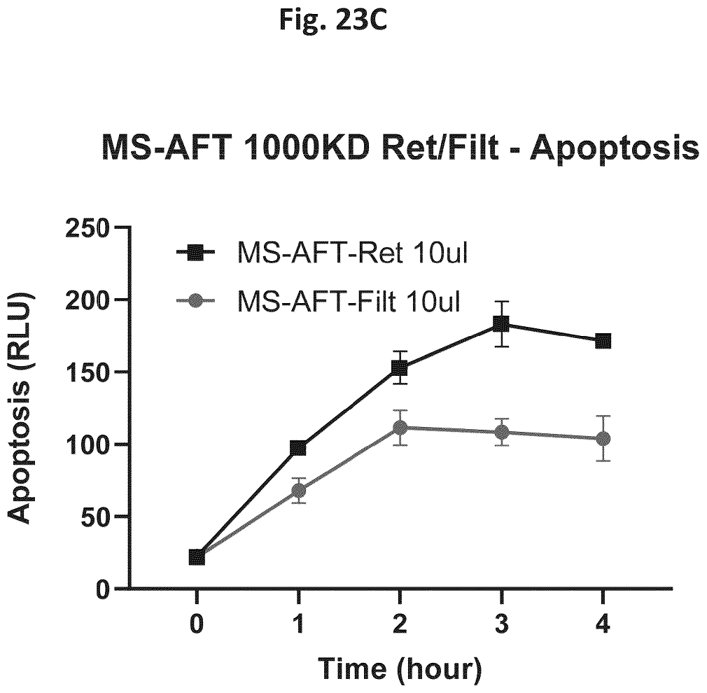

In another exemplary embodiment, MS and HC plasma samples were diluted at 1:30 in PBS and centrifuged through a 0.1 μm microfilter tube (Millipore, Ultrafree®-MC Filter #UFC30VV25). The retentates and the filtrates were collected for analysis. FIG. 23A shows that in SH-SY5Y cells, 0.1 μm MS plasma retentates caused significantly higher apoptosis than HC plasma retentates (****p<0.0001, n=8). The 0.1 μm filtrates from MS and HC plasma did not exhibit cytotoxicity. As a comparison, MS A-FT also led to significantly higher apoptosis than HC A-FT (****p<0.0001). The filtrates and plasma alone, however, showed no cytotoxicity. The data shown in FIG. 23B were obtained by using a human IgG subclass ELISA kit to quantify the IgG concentrations in the retentates. There was a significantly higher IgG1 level in MS retentates compared to HC Retentates (****p<0.0001). MS-AFT samples were centrifuged through a 1000 kDa microfilter tube (Sartorius Vivaspin 500, VS0161), after which the retentates and filtrates were used for cytotoxicity tests in SH-SY5Y cells. The results, shown in FIG. 23C, showed that the retentates indeed induced greater levels of apoptosis than the filtrates, confirming that plasma extracted from MS patients contain elevated levels of IgG1 and contain particles (>100 nm) produce significantly higher levels of neuronal cytotoxicity related to plasma obtained from healthy controls.

### Example 25

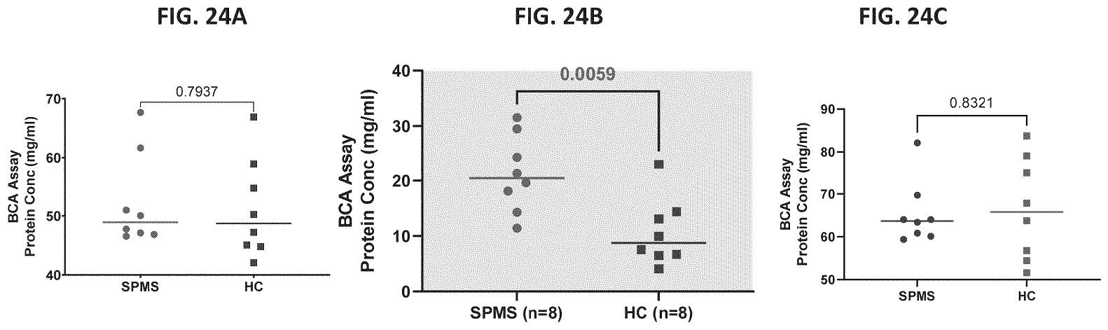

In another exemplary embodiment, MS and HC plasma samples were diluted at 1:30 in PBS and centrifuged through a 0.1 μm microfilter tube (Millipore, Ultrafree®-MC Filter #UFC30VV25). The retentates and the filtrates were collected for analysis. Protein concentrations were determined by Pierce BCA assay kit (Thermo 23227). As shown in FIG. 24A, protein concentrations were similar in MS and HC plasma filtrates (p=0.79, n=8), but as shown in FIG. 24B, MS plasma 0.1 μm retentates had significantly higher protein concentrations than HC plasma retentates (p=0.0059, n=8). FIG. 24C shows that total protein concentrations in the MS and HC plasma were similar (p=0.83, n=8).

### Example 26

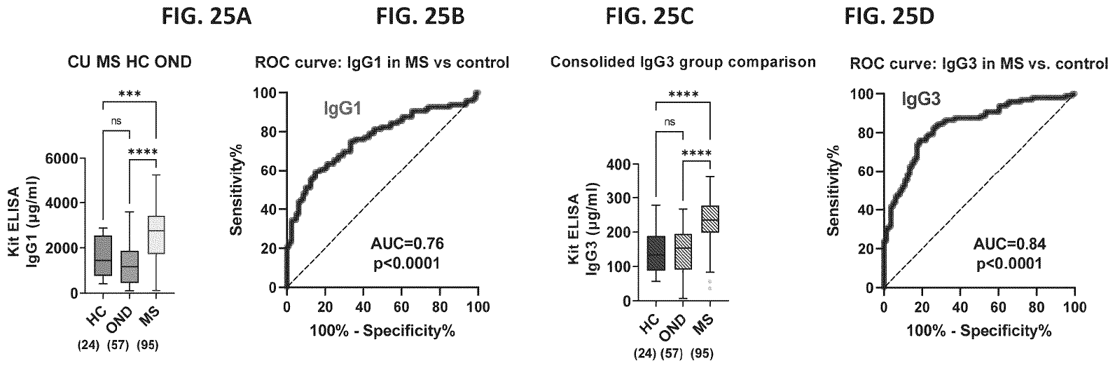

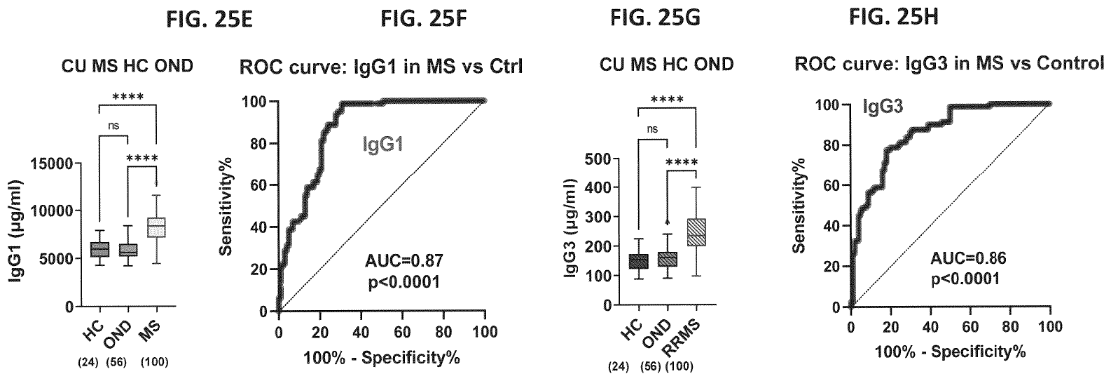

In another exemplary embodiment, a commercial kit (Invitrogen 991000) (FIGS. 25A-D) and an adapted ELISA assay disclosed herein (FIGS. 25E-H) were used to independently determine IgG1 and IgG3 levels present within MS and control A-FT samples derived from serum. As shown in FIG. 25A, the ELISA Kit assay revealed that MS A-FT had significantly higher levels of IgG1 than HC A-FT (***p<0.001) and OND (other neurological diseases (****p<0.0001). The ROC curve (FIG. 25B) of the IgG1 ELISA kit had an area of 0.76 (p<0.0001), and as shown in FIG. 25C, the ELISA kit also detected significantly higher levels of IgG3 in MS A-FT than in HC A-FT (****p<0.0001) and OND (****p<0.0001). The ROC curve (FIG. 25D) of the IgG3 ELISA kit had an area of 0.84 (p<0.0001). As shown in FIG. 25E, the disclosed, custom IgG1 ELISA assay revealed that MS A-FT had significantly higher levels of IgG1 than HC A-FT (****p<0.0001) and OND (****p<0.0001). The ROC curve (FIG. 25F) of the disclosed IgG1 ELISA had an area of 0.87 (p<0.0001), and as shown in FIG. 25G, the disclosed IgG3 ELISA assay revealed MS A-FT had significantly higher levels of IgG3 than HC A-FT (****p<0.0001) and OND (****p<0.0001). The ROC curve (FIG. 25H) of the disclosed IgG3 ELISA had an Area of 0.86 (p<0.0001). Accordingly, the custom ELISA assays and associated components disclosed herein may produce results at least equally reliable as commercially available ELISA kits, but advantageously without requiring the specialized components and reagents purchased with such kits.

### Example 27

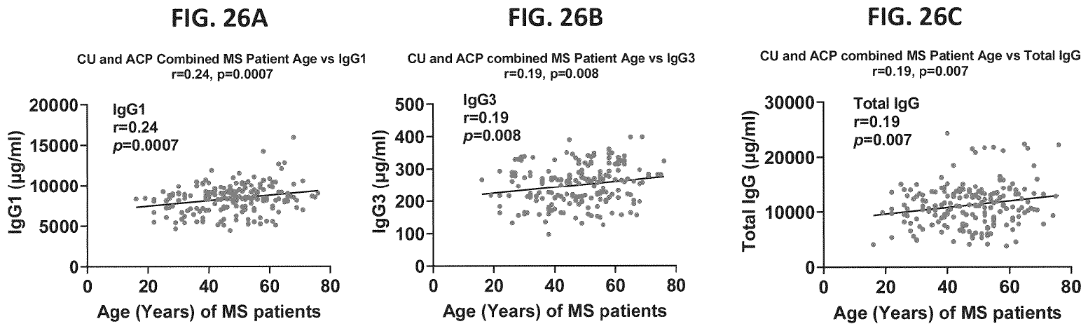

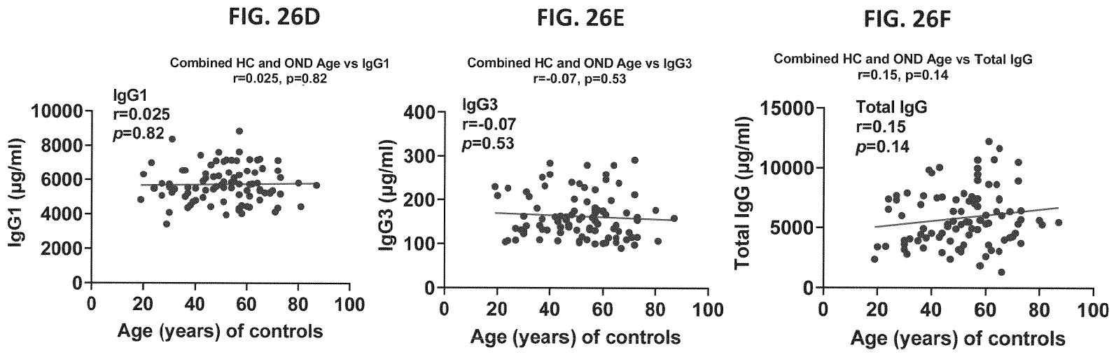

In another exemplary embodiment, MS patients' ages (FIGS. 26A-C, n=190) and control subjects' ages (FIGS. 26D-F, n=90) were plotted against their corresponding IgG1, IgG3, and total IgG levels determined in accordance with embodiments disclosed herein. The lineal line was plotted and correlation analyses performed on the two parameters. The r and p values are shown in each graph. As shown in FIGS. 26A-C, in combined MS groups, IgG1, IgG3, and total IgG levels showed a significant correlation trend between IgG and age (p<0.01). As depicted in FIGS. 26D-F, plotting IgG1, IgG3, and total IgG data for combined control groups revealed no significant differences between age and IgG levels (p>0.1). Subject age, among other factors, may thus be factored into MS diagnoses made using IgG levels.

### Exemplary Methods

The exemplary methods described above were performed using one or more of the following techniques, which may be implemented in whole or in part to diagnose MS, monitor MS over time and in response to therapy, and/or distinguish between MS subtypes. One or more of the following techniques may be performed in whole or in part using one or more kits, which may be portable and/or compatible with standard laboratory equipment and supplies, e.g., pipettes, centrifuge tubes, centrifuges, buffers, etc.:

Preparation of Protein A and Protein G Flow-Through. The techniques, reagents, and equipment used to collect Protein A flow-through (“A-FT”) and Protein G flow-through (“G-FT”) from serum and/or plasma samples may vary. In one example, Pierce™ Protein A-coated 96-well plates (Thermo #15130) were used for collection of the flow-through. Plasma and CSF samples diluted in PBS (Gibco #10010-023) at 1:100 dilutions were used for obtaining the flow-through. The plates were rinsed twice with 1×PBS, and 200 μl of 1:100 diluted sera/plasma samples were added to the wells of the plates. After thorough mixing, the plates were sealed and immediately incubated at 4° C. for 2-5 hours. The unbound solution/supernatant named #1 Protein A flow-through (A-FT) was transferred to a second pre-rinsed Protein A plate and incubated overnight at 4° C. The flow-through collected was named #2 A-FT. Similar procedures were carried for collecting #3 and the final flow-through except that the incubation times were 2 hours each. The final A-FT was collected, aliquoted, and stored at −80° C. Similarly, the 1:100 diluted plasma samples were incubated 4 consecutive times with Protein G coated 96-well plate (Thermo #15131), and the final supernatant was collected as Protein G Flow-through (G-FT).

Protein A-coated plates may be used for procedures in which high throughput analysis is advantageous. Additional embodiments may use smaller or larger plates coated with Protein A, or different Protein A substrates altogether, e.g., Protein A-coated centrifuge tubes or columns. Initial sample dilutions may also vary, ranging from 1:100 down to 1:10 or up to 1:1000. The number of plate rinsing steps may also vary, ranging from one to two, three, four, five or more. Incubation times and temperatures may also vary, provided the integrity of the IgG aggregates is preserved. The number of distinct A-FTs generated may also vary. For example, some embodiments may only require a single Protein A binding step, such that the first-collected A-FT may be immediately analyzed for total IgG. IgG1, and/or IgG3 content without exposing the A-FT to additional Protein A capture compositions.

Kit IgG subclass ELISA. A variety of ELISA methods and kits can be utilized to determine IgG concentrations in accordance with embodiments described herein. In some examples, a human IgG subclass ELISA kit (Invitrogen #991000) can be used to determine the concentrations of all IgG subclasses (IgG1-4) in plasma and A-FT samples. The A-FT and sera samples pre-diluted at 1:100 in PBS were further diluted at 1:30 using the dilution buffer provided by the kits (total dilution 1:3,000). 50 μl of corresponding antibody (MAB anti-hIgG1, 2, 3, 4) were added to each well, followed by addition of 50 μl of diluted samples or standards. The plate was incubated for 1 hour at room temperature with shaking (25 rpm). After 6 times of washing, the plate was incubated with HRP-anti-human IgG (1:50 dilution) followed by TMB substrate incubation. The color intensity was determined by a microplate reader (BioTek Synergy H₂ with Gen5 1.11 software).

Western blots. Mini-PROTEAN® TGX 4-15% Gels (BioRad) were used for SDS-PAGE analysis with 1× Tris/Glycine/SDS running buffer (BioRad). CSF and paired plasma (1 μg total IgG antibody equivalent) in TBS were denatured and reduced by incubation with 1× lane marker reducing sample buffer containing dithiothreitol (Thermo Scientific) at 95° C. for 10 minutes. Gels were electrophoresed for 40 minutes at a constant voltage of 150V, and electro-blotted onto PVDF membranes (Bio-Rad) for 45 minutes at a constant 10 V using Trans-Blot® Semi-Dry Cell (Bio-Rad). Membranes were blocked overnight in 1× casein/TBS/0.05% Tween 20 (Vector Labs). Triplicate blots were probed with corresponding antibodies for total IgG, IgG1, and IgG3 detection. HRP-conjugated goat anti-human IgG (H+L) (Vector Labs) was used for total IgG detection at a dilution of 1:2000. Monoclonal mouse anti-human IgG1 (1:2000 dilution, clone 8c/6-39, Sigma), and monoclonal mouse anti-human IgG3 (1:2000 dilution, clone HP-6050, Sigma) were incubated with membranes at 4° C. overnight. SuperSignal® West Pico was used for total IgG detection, and anti-mouse IgG (H+L) HRP/West Femto for detection of IgG1 & IgG3).

Adapted developed human IgG1 and IgG3 ELISA procedures. To detect human IgG1 and IgG3 levels in the Protein A-flow-through (A-FT) or Protein G-flow-through (G-FT) from patient plasma samples, an adapted ELISA assay was developed. The MaxiSorp treated 96-well plate (white, flat bottom, Thermo #436110) was used for adapted ELISA assays. The plate was rinsed twice with 1×TBST (diluted from Bio-Rad 10×TBS #1706435 with MilliQ water, with 0.1% Tween-20). Then the plate was coated with 100 μl/well of mouse anti-human IgG1 antibody (Sigma #12513, final concentration 10 μg/ml for detecting IgG1) or goat anti-human IgG (H+L) antibody (Vector Lab #AI-3000, final concentration 50 μg/ml for detecting IgG3), diluted in 0.1 M NaHCO₃, pH 9.4 and incubated at 4° C. refrigerator overnight. The next day, the coating solution was removed and blocked with 300 μl/well of 3% BSA in TBS for 5 hours at room temperature with orbital shaking (25 rpm). The plate was rinsed 3 times with 1×TBST. The A-FT or G-FT samples had been prepared as described above, aliquoted at 45 μl/well in PCR stripe tubes, and stored at −80° C. freezer. The samples were thawed on ice and diluted at 1:100 with 1×TBS in a 96-well U-bottom plate (Greiner Bio-One #60180-P113). For each well, 50 μl of the diluted A-FT or G-FT samples (or 50 μl of standards) and 50 μl of TBS were added to the ELISA plate, followed by incubation at 4° C. cold room with shaking (25 rpm) overnight. The next day, the samples were removed and the plate was washed 6 times with 1× TBST. Biotinylated goat anti-human IgG-Fc antibody (Rockland #609-1603, 1:3,000 diluted in 1×TBS, for detecting hIgG1) or with biotinylated mouse anti-human IgG3 antibody (Southem Biotech #9210-08, diluted at 1:3,000 in 1×TBS) was added to the plate at amounts of 100 μl/well. The plate was then incubated at room temperature for 1 hour with shaking. After that, the plate was washed 6 times with 1×TBST, followed by incubation with NeutrAvidin-HRP (Thermo #31030, diluted 1:10,000 in 1×TBS, 100 μl/well for both IgG1 and IgG3 plates) at room temperature for 1 hour with shaking. Finally, the ELISA plate was washed 6 times with 1×TBST, and then developed with TMB substrate (SeraCare two components substrate kit, #5120-0047, 1:1 mixture. 100 μl/well) for 15-30 min. The reaction was stopped with 0.1 N HCl, and the color intensity was measured by a microplate reader (BioTek Synergy plate reader).

One or more of the parameters of the adapted ELISA procedure for human IgG1, IgG3 or total IgG may vary in different embodiments. For example, the per-well concentration of mouse anti-human IgG1 and/or goat anti-human IgG antibody may vary, ranging from 100 μl/well down to 10 μl/well, 20 μl/well, 30 μl/well, 40 μl/well, 50 μl/well, 60 μl/well, 70 μl/well, 80 μl/well, 90 μl/well, or above 100 μl/well, such as 110 μl/well, 120 μl/well, 130 μl/well, 140 μl/well, 150 μl/well, or greater.

ELISA procedure for Protein A or Protein G captured plasma antibody for the detection of total IgG. After the completion of Protein A-Flow-through (A-FT) or Protein G-Flow-though (G-FT) preparation procedure (as described above), the IgG-bounded wells were added with 200 μl of PBS and stored at 4° C. refrigerator. To detect the levels of these captured IgG in Protein A or Protein G plate, an ELISA assay was developed as follows. The plate was washed 3 times with 1×TBST, followed by 300 μl/well of blocking buffer (3% BSA in 1×TBS) for 1 hour at room temperature with shaking (25 rpm). The plate was rinsed 3 times with 1× TBST. Then the plate was added with 100 μl/well of the biotinylated goat anti-human IgG-Fc antibody (Rockland #609-1603, 1:3,000 diluted in 1×TBS) and incubated at room temperature for 1 hour with shaking. After that, the plate was washed 6 times with 1×TBST, followed by incubation with NeutrAvidin-HRP (Thermo #31030, diluted 1:20,000 in 1×TBS, 100 μl/well) at room temperature for 1 hour with shaking. Finally, the ELISA plate was washed 6 times with 1×TBST, and then developed with TMB substrate (SeraCare two components substrate kit, #5120-0047, 1:1 mixture, 100 μl/well) for 10-15 min. The reaction was stopped with 0.1 N HCl, and the color intensity was measured by a microplate reader (BioTek Synergy plate reader).

ELISA procedure for goat anti-human IgG-Fc antibody, Fc-III peptide, or goat anti-human IgG (H+L) coated plate for the detection of total human IgG from patient's plasma. Goat anti-human IgG-Fc antibody, Fc-III peptide, or goat anti-human IgG (H+L) coated plates were used to capture IgG from plasma samples and then detect the total IgG levels using ELISA assay. The MaxiSorp treated 96-well plate (white, flat bottom, Thermo #436110) was used for antibody or peptide coating. The plate was rinsed twice with 1×TBST (diluted from Bio-Rad 10×TBS #1706435 with MilliQ water, with 0.1% Tween-20). Then the plate was coated with 100 μl/well of goat anti-human IgG-Fc antibody (Rockland #609-1103, final concentration 10 ug/ml), synthetic Fc-III peptide (Amino Acid Sequence: H2N-DCAWHLGELVWCT-OH, New England Peptide, final concentration 100 μg/ml), or goat anti-human IgG (H+L) (Vector Lab #AI-3000, final concentration 50 ng/ml), all diluted in 0.1 M NaHCO3, pH 9.4 and incubated at 4° C. refrigerator overnight. The next day, the coating solution was removed and blocked with 300 μl/well of 3% BSA in TBS for 5 hours at room temperature with shaking (25 rpm). The plate was rinsed 3 times with 1×TBST. The 1:100 diluted plasma samples had been aliquoted at 45 μl/well in PCR stripe tubes and stored at −80° C. freezer. The samples were thawed on ice and diluted further at 1:100 with 1×TBS in a 96-well U-bottom plate (Greiner Bio-One #60180-P113). For each well, 50 μl of the diluted samples (or 50 μl of standards) and 50 μl of TBS were added to the ELISA plate, followed by incubation at 4° C. cold room with shaking (25 rpm) overnight. The next day, the samples were removed, and the plate was washed 6 times with 1×TBST. Then the plate was added with 100 μl/well of the biotinylated goat anti-human IgG (H+L) antibody (Vector Lab #BA3000, 1:3,000 diluted in 1×TBS for anti-Fc and Fc-III coated plates), or biotinylated goat anti-human IgG-Fc antibody (Rockland #609-1603, 1:3,000 diluted in 1×TBS for anti-IgG (H+L) coated plate), and incubated at room temperature for 1 hour with shaking. After that, the plate was washed 6 times with 1×TBST, followed by incubation with NeutrAvidin-HRP (Thermo #31030, diluted 1:10,000 in 1×TBS. 100 μl/well) at room temperature for 1 hour with shaking. Finally, the ELISA plate was washed 6 times with 1×TBST, and then developed with TMB substrate (SeraCare two components substrate kit, #5120-0047, 1:1 mixture, 100 μl/well) for 10-20 min. The reaction was stopped with 0.1 N HCl, and the color intensity was measured by a microplate reader (BioTek Synergy plate reader).

A-FT binding with Fc-III peptide, followed by ELISA assay using goat anti-IgG-Fc antibody-coated plate for the detection of IgG3. Synthetic Fc-III peptide was used to bind A-FT, and the binding mixture was used for ELISA procedure with goat anti-human IgG-Fc antibody-coated plate to detect total IgG levels. The MaxiSorp treated 96-well plate (white, flat bottom. Thermo #436110) was used for antibody coating. The plate was rinsed twice with 1×TBST (diluted from Bio-Rad 10×TBS #1706435 with MilliQ water, with 0.1% Tween-20). Then the plate was coated with 100 μl/well of goat anti-human IgG-Fc antibody (Rockland #609-1103, final concentration 10 μg/ml) diluted in 0.1 M NaHCO₃, pH 9.4, and incubated at 4° C. refrigerator overnight. The next day, the coating solution was removed and blocked with 300 μl/well of 3% BSA in TBS for 5 hours at room temperature with shaking (25 rpm). The plate was rinsed 3 times with 1×TBST. The A-FT (5 μl, about 2 μg of total proteins) was mixed with 25 μl of Fc-III peptide (1-20 μg Fc-III, Amino Acid Sequence: H2N-DCAWHLGELVWCT-OH, New England Peptide) for 1 hour at room temperature. Then 3 μl of AFT-Fc-III mixture ( 1/10 of the total mixture) was added to 97 μl of 1×TBS, and transferred to each well, and incubated for 2 hours at room temperature with shaking. The samples were removed, and the plate was washed 6 times with 1×TBST. Then the plate was added with 100 μl/well of the biotinylated mouse anti-human IgG3 antibody (Southern Biotech #9210-08, 1:3,000 diluted in 1×TBS) and incubated at room temperature for 1 hour with shaking. After that, the plate was washed 6 times with 1×TBST, followed by incubation with NeutrAvidin-HRP (Thermo #31030, diluted 1:10,000 in 1×TBS, 100 μl/well) at room temperature for 1 hour with shaking. Finally, the ELISA plate was washed 6 times with 1×TBST, and then developed with TMB substrate (SeraCare two components substrate kit, #5120-0047, 1:1 mixture, 100 μl/well) for 10-20 min. The reaction was stopped with 0.1 N HCl, and the color intensity was measured by a microplate reader (BioTek Synergy plate reader).

ELISA procedure for mouse anti-human IgG-Kappa chain antibody or mouse anti-human IgG-Lamda chain antibody-coated plate for the detection of total human IgG from patient's plasma. Mouse anti-human IgG-Kappa chain antibody or mouse anti-human IgG-Lamda chain antibody-coated plates were used to capture IgG from plasma samples and then detect the total IgG levels using ELISA assay. The MaxiSorp treated 96-well plate (white, flat bottom, Thermo #436110) was used for antibody or peptide coating. The plate was rinsed twice with 1×TBST (diluted from Bio-Rad 10×TBS #1706435 with MilliQ water, with 0.1% Tween-20). Then the plate was coated with 100 μl/well of mouse anti-human IgG-Kappa chain antibody (Sigma #K4377, final concentration 10 μg/ml) or mouse anti-human IgG-Lamda chain antibody (Raybio #188-10939, final concentration 10 μg/ml) diluted in 0.1 M NaHCO₃, pH 9.4 and incubated at 4° C. refrigerator overnight. The next day, the coating solution was removed and blocked with 300μ/well of 3% BSA in TBS for 5 hours at room temperature with shaking (25 rpm). The plate was rinsed 3 times with 1×TBST. The 1:100 diluted plasma samples had been aliquoted at 45 μl/well in PCR stripe tubes and stored at −80° C. freezer. The samples were thawed on ice and diluted further at 1:5 with 1×TBS in a 96-well U-bottom plate (Greiner Bio-One #60180-P113). For each well, 50 μl of the diluted samples (or 50 μl of standards) and 50 μl of TBS were added to the ELISA plate, followed by incubation at room temperature for 1 hour with shaking (25 rpm). After that, the samples were removed, and the plate was washed 6 times with 1×TBST. Then the plate was added with 100 μl/well of the biotinylated goat anti-human IgG-Fc antibody (Rockland #609-1603, 1:3,000 diluted in 1× TBS), and incubated at room temperature for 1 hour with shaking. After that, the plate was washed 6 times with 1×TBST, followed by incubation with NeutrAvidin-HRP (Thermo #31030, diluted 1:10,000 in 1×TBS, 100 μl/well) at room temperature for 1 hour with shaking. Finally, the ELISA plate was washed 6 times with 1×TBST, and then developed with TMB substrate (SeraCare two components substrate kit, #5120-0047, 1:1 mixture, 100 μl/well) for 10-20 min. The reaction was stopped with 0.1 N HCl, and the color intensity was measured by a microplate reader (BioTek Synergy plate reader).

Using 300KD molecular weight cut off filter tube to separate A-FT proteins. The 300 KDa molecular weight cut-off filter tube (Pall Life Science, Nanosep 300KD, Omega, #OD300C34) was used to separate A-FT and plasma proteins. Before loading the protein samples, the filter tube was wetted with 0.5 ml of PBS (Ca—Mg-Free PBS, Invitrogen #10010023) and incubated at room temperature for 5 min. Then the tubes were centrifuged at 6,000 g for 10 min. The PBS in the collection tube was discarded. The A-FT or diluted plasma samples were loaded to the tube (80 μl of A-FT diluted with 240 μl PBS, total 320 μl), incubated for 5 min at room temperature, followed by centrifugation at 6,000 g for 10 min. The fraction in the top sample tube (“retentate”) was collected, about 60 μl. The fraction in the bottom collection tube (“filtrate”) was also collected, about 240 μl. Both fractions were stored at −80° C. for late analysis.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1-4. (canceled)

5. A method for diagnosing a neurological disorder in a subject, comprising:
obtaining one or more serum or plasma samples from a subject suspected of having or developing a neurological disorder;
exposing the one or more serum samples or plasma to one or more of a Protein A matrix, a Protein G matrix, or a Protein A/Protein G matrix;
collecting a flow-through or unbound portion of the one or more serum or plasma samples after exposing the samples to the one or more of the Protein A matrix, the Protein G matrix, or the Protein A/Protein G matrix;
measuring IgG levels in the flow-through or unbound portion of the one or more serum or plasma samples; and
diagnosing a neurological disorder in the subject based on at least one of a total IgG (H+L)/Fc level, an IgG1 level, an IgG3 level, kappa light chain level, or lambda light chain level in the flow-through or unbound portion of the one or more serum or plasma samples compared to one or more samples from a control subject not having a neurological disorder.

6. The method according to claim 5, comprising diagnosing the subject with MS based on the IgG1 level in the flow-through of the one or more serum samples.

7. (canceled)

8. The method according to claim 5, wherein measuring IgG (H+L)/Fc levels comprises measuring total IgG (H+L)/Fc and comparing total IgG (H+L)/Fc to healthy control subject samples, and wherein elevated total IgG (H+L)/Fc levels compared to the healthy control subject samples is indicative of having a neurological disorder in the subject.

9. The method according to claim 5, wherein measuring IgG levels comprises using an ELISA assay, flow cytometry, Nephelometry or other immunoassay for detecting IgG antibodies in the flow-through of the one or more serum samples.

10. The method according to claim 5, wherein the method distinguishes a subject having MS from a subject having a different inflammatory CNS disorder based on the level of IgG or the level of IgG1.

11. The method according to claim 5, wherein the one or more serum samples are exposed to one or more of the Protein A matrix, the Protein G matrix, or the Protein A/Protein G matrix at least two times by collecting the flow-through and reapplying the flow-through to at least a second Protein A matrix, Protein G matrix, or Protein A/Protein G matrix.

12. The method according to claim 7, wherein the one or more serum samples are exposed to a Protein A matrix and further comprising measuring IgG3 levels in the flow-through, wherein elevated IgG3 levels compared to healthy control samples and samples from a subject having a neurological disorder other than MS is indicative that the subject has MS.

13. The method according to claim 7, wherein the one or more serum samples are exposed to a Protein G matrix and further comprising measuring IgG1 levels in the flow-through of the one or more serum samples, wherein elevated IgG1 levels compared to healthy control samples and samples from a subject having a neurological disorder other than MS is indicative that the subject has MS.

14. The method according to claim 5, further comprising performing a neuronal cytotoxic analysis of one or more serum samples exposed to at least one of a Protein A or Protein G matrix and measuring cytotoxicity, wherein increased cytotoxicity, as demonstrated by increased apoptosis, necrosis, or necroptosis in primary neuronal cells or cell lines selected from neurons, astrocytes, oligodendrocytes, microglia, or mouse brain tissues in the samples is indicative of at least one of MS or MS progression.

15. The method according to claim 5, further comprising exposing the flow-through to a filter having a molecular weight cut-off of about 110 kDa, about 200 kDa or about 300 kDa or size cut-off of about 100 nm and further comprising measuring at least one of IgG1 or IgG4 levels in a retentate.

16. The method according to claim 5, further comprising treating the subject to reduce IgG levels, wherein reducing IgG1 in the subject treats MS in the subject.

17. (canceled)

18. A method for identifying MS (Multiple Sclerosis) subtypes in a subject comprising,
obtaining one or more serum samples from a subject suspected of having or developing MS;
exposing the one or more serum samples to a Protein A matrix;
collecting flow-through of the one or more serum samples after exposing the samples to the Protein A matrix;
measuring IgG levels in the flow-through of the one or more serum samples;
comparing the IgG levels in the flow-through of the serum samples to IgG levels in flow-through of control samples;
diagnosing Relapsing-Remitting MS (RRMS), Secondary-Progressive MS (SPMS), or Primary-Progressive MS (PPMS) in the subject based on the IgG levels in the flow-through of the one or more serum samples; and
adjusting a treatment of the subject based on the diagnosis.

19. The method according to claim 18, wherein measuring IgG levels comprises measuring IgG1 levels, and further comprising diagnosing the subject with Secondary-Progressive MS (SPMS) when the level of IgG1 is elevated compared to an IgG1 level of a control sample or unprocessed sample.

20. The method according to claim 18, wherein the flow-through of the one or more serum samples is further subjected to mass spectrometry.

21. The method according to claim 18, further comprising analyzing the flow-through of the one or more serum samples for protein expression of one or more of IGKV1-5 (Immunoglobulin Kappa Variable 1-5); IGLV2-18 (Immunoglobulin Lambda Variable 2-18); C5 (Complement component 5); CFI (Complement Factor I); ORM1 (Orosomucoid 1); IGHV1-18 (Immunoglobulin Heavy Variable 1-18); IGHV3-49 (Immunoglobulin Heavy Variable 3-49); IGLV3-21 (Immunoglobulin Lambda Variable 3-21); LGALS3BP (Galectin 3 Binding Protein); PROC (Protein C, Inactivator Of Coagulation Factors Va And VIIIa); and SERPINAS (serine proteinase inhibitor).

22-23. (canceled)

24. The method according to claim 20, further comprising identifying clusters of differentially expressed proteins using data obtained from the mass spectrometry.

25. A method for diagnosing a neurological disorder in a subject, comprising:
obtaining one or more serum samples from a subject suffering from, suspected of having, or suspected of developing a neurological disorder;
processing the one or more serum samples to create an enriched sample having a greater concentration of IgG aggregates than an unprocessed serum sample;
combining the enriched sample with a population of live neuronal cells;
quantifying the number of neuronal cells that die in the presence of the enriched sample to create a cytotoxicity value;
assessing the concentration of IgG aggregates in the enriched sample from the cytotoxicity value, and thereby diagnosing a neurological disorder in a subject.

26. The method of claim 25, wherein processing step includes enriching for IgG aggregates greater than about 110 kDa, about 200 kDa, or about 300 kDa.

27. The method of claim 26, wherein the processing step includes passing the serum sample through a filter.

28. The method of claim 25, wherein the neurological disorder is selected from Relapsing-Remitting MS (RRMS), Secondary-Progressive MS (SPMS), or Primary-Progressive MS (PPMS).
